SEQUENCE LISTING (16 sequences)

SEQ ID NO: 1 - DNA, 636 bp, Unknown
  FGF-21
atggctcccttagccgaagtcgggggctttctgggcggcctggagggcttgggccagcaggtgggttcgcatttcctgttgcctcctgccggggagcggccgccgctgctgggcgagcgcaggagcgcggcggagcggagcgcgcgcggcgggccgggggctgcgcagctggcgcacctgcacggcatcctgcgccgccggcagctctattgccgcaccggcttccacctgcagatcctgcccgacggcagcgtgcagggcacccggcaggaccacagcctcttcggtatcttggaattcatcagtgtggcagtgggactggtcagtattagaggtgtggacagtggtctctatcttggaatgaatgacaaaggagaactctatggatcagagaaacttacttccgaatgcatctttagggagcagtttgaagagaactggtataacacctattcatctaacatatataaacatggagacactggccgcaggtattttgtggcacttaacaaagacggaactccaagagatggcgccaggtccaagaggcatcagaaatttacacatttcttacctagaccagtggatccagaaagagttccagaattgtacaaggacctactgatgtacacttga

SEQ ID NO: 2 - protein, 211 aa, Unknown
  FGF-21
MAPLAEVGGFLGGLEGLGQQVGSHFLLPPAGERPPLLGERRSAAERSARGGPGAAQLAHLHGILRRRQLYCRTGFHLQILPDGSVQGTRQDHSLFGILEFISVAVGLVSIRGVDSGLYLGMNDKGELYGSEKLTSECIFREQFEENWYNTYSSNIYKHGDTGRRYFVALNKDGTPRDGARSKRHQKFTHFLPRPVDPERVPELYKDLLMYT

SEQ ID NO: 3 - DNA, 513 bp, Unknown
  FGF-23
atgcgccgccgcctgtggctgggcctggcctggctgctgctggcgcgggcgccggacgccgcgggaaccccgagcgcgtcgcggggaccgcgcagctacccgcacctggagggcgacgtgcgctggcggcgcctcttctcctccactcacttcttcctgcgcgtggatcccggcggccgcgtgcagggcacccgctggcgccacggccaggacagcatcctggagatccgctctgtacacgtgggcgtcgtggtcatcaaagcagtgtcctcaggcttctacgtggccatgaaccgccggggccgcctctacgggtcgcgactctacaccgtggactgcaggttccgggagcgcatcgaagagaacggccacaacacctacgcctcacagcgctggcgccgccgcggccagcccatgttcctggcgctggacaggaggggggggccccggccaggcggccggacgcggcggtaccacctgtccgcccacttcctgcccgtcctggtctcctga

SEQ ID NO: 4 - protein, 170 aa, Unknown
  FGF-23
MRRRLWLGLAWLLLARAPDAAGTPSASRGPRSYPHLEGDVRWRRLFSSTHFFLRVDPGGRVQGTRWRHGQDSILEIRSVHVGVVVIKAVSSGFYVAMNRRGRLYGSRLYTVDCRFRERIEENGHNTYASQRWRRRGQPMFLALDRRGGPRPGGRTRRYHLSAHFLPVLVS

SEQ ID NO: 5 - protein, 208 aa, Unknown
  FGF-9
MAPLGEVGNYFGVQDAVPFGNVPVLPVDSPVLLSDHLGQSEAGGLPRGPAVTDLDHLKGILRRRQLYCRTGFHLEIFPNGTIQGTRKDHSRFGILEFISIAVGLVSIRGVDSGLYLGMNEKGELYGSEKLTQECVFREQFEENWYNTYSSNLYKHVDTGRRYYVALNKDGTPREGTRTKRHQKFTHFLPRPVDPDKVPELYKDILSQS

SEQ ID NO: 6 - protein, 207 aa, Unknown
  FGF-16
MAEVGGVFASLDWDLHGFSSSLGNVPLADSPGFLNERLGQIEGKLQRGSPTDFAHLKGILRRRQLYCRTGFHLEIFPNGTVHGTRHDHSRFGILEFISLAVGLISIRGVDSGLYLGMNERGELYGSKKLTRECVFREQFEENWYNTYASTLYKHSDSERQYYVALNKDGSPREGYRTKRHQKFTHFLPRPVDPSKLPSMSRDLFHYR

SEQ ID NO: 7 - protein, 117 aa, Unknown
  FGF-22
  MISC_FEATURE (1)..(1): Xaa may be any amino acid
XGMLASYSVAVAMVTTRGVASRLYLDSNHKGDLYASVRLAQESVFWGQSEENWSYTHSSNLYKHVDTRRRYYVPLNQGATPSAGTRSLRRQNYTHVLPRPVDPDKVPELYKDILSQS

SEQ ID NO: 8 - protein, 208 aa, Xenopus laevis
MAPLADVGTFLGGYDALGQVGSHFLLPPAKDSPLLFNDPLAQSERLSRSAPSDLSHLQGILRRRQLYCRTGFHLQILPDGNVQGTRQDHSRFGILEFISVAIGLVSIRGVDTGLYLGMNDKGELFGSEKLTSECIFREQFEENWYNTYSSNLYKHGDSGRRYFVALNKDGTPRDGTRAKRHQKFTHFLPRPVDPEKVPELYKDLMGYS

SEQ ID NO: 9 - protein, 4 aa, Artificial
  Peptide
LYGS

SEQ ID NO: 10 - protein, 4 aa, Artificial
  Peptide
HFLP

SEQ ID NO: 11 - protein, 5 aa, Artificial
  Peptide
VQGTR

SEQ ID NO: 12 - protein, 10 aa, Artificial
  Peptide
RIEENGHNTY

SEQ ID NO: 13 - protein, 10 aa, Artificial
  Peptide
QFEENWYNTY

SEQ ID NO: 14 - protein, 6 aa, Artificial
  Peptide
AGTPSA

SEQ ID NO: 15 - protein, 6 aa, Artificial
  Peptide
AAERSA

SEQ ID NO: 16 - protein, 6 aa, Artificial
  6X His tag
HHHHHH

(19) United States
(12) Patent Application Publication
(10) Pub. No.: US 2008/0057076 A1
(43) Pub. Date: Mar. 6, 2008

(54) Novel fibroblast growth factors

(75) Inventors: Peter W. Bringmann, Concord, CA (US); Daryl Faulds, Mill Valley, CA (US); Branislava Mitrovic, Walnut Creek, CA (US); Subha Srinivasan, Greenbrae, CA (US)

Correspondence Address: BAYER HEALTH CARE, 800 DWIGHT WAY, BERKELEY, CA, 94710, US

(73) Assignee: Schering Aktiengesellschaft, Berlin (DE)

(21) Appl. No.: 11/821,191

(22) Filed: Jun. 22, 2007

Related U.S. Application Data
(63) Continuation of application No. 10/005,646, filed on Dec. 7, 2001, now abandoned.
(60) Provisional application No. 60/251,837, filed on Dec. 8, 2000.

(51) Int. Cl. A61K 39/00; A61K 31/7088; A61P 25/00; A61P 37/06; A61K 38/18

(52) U.S. Cl. 424/185.1; 514/12; 514/44

(57) ABSTRACT

Novel nucleic acids, polypeptide sequences, and nucleic acid regulators thereof, have been identified which code for a fibroblast growth factor (FGF), preferably FGF-20 or FGF-23, a class of polypeptides involved in development, differentiation, and morphogenesis, e.g., in cell-cell signalling and cell proliferation. An FGF of the present invention, fragments thereof, and derivatives thereof, have one or more of the following biological activities, e.g., promoting wound healing; promoting neuronal survival; stimulating cell proliferation, e.g., proliferation of stem cells, fibroblasts, neurons, glia, oligodendrocytes, Schwann cells, or progenitors thereof; modulating differentiation of cells; inducing embryonic development; stimulating neurite outgrowth; enhancing recovery from nerve or neuronal damage; stimulating myelination; stimulating angiogenesis; receptor binding activity; modulating tumorigenesis, etc.

This application claims priority of Provisional application 60/251,837, filed Dec. 8, 2000, which is incorporated by reference herein in its entirety.

### BACKGROUND OF THE INVENTION

Fibroblast growth factors play an important role in variety of biological functions, including, e.g., cell proliferation and differentiation, and development.

### DESCRIPTION OF THE INVENTION

Novel nucleic acids, polypeptide sequences, and nucleic acid regulators thereof, have been identified which code for a fibroblast growth factor (FGF), preferably FGF-20 (called FGF-21 in the provisional application corresponding to this application) or FGF-23 (which is the same as published FGF-22), a class of polypeptides involved in development; differentiation, and morphogenesis, e.g., in cell-cell signalling and cell proliferation. An FGF of the present invention, fragments thereof, and derivatives thereof, have one or more of the following biological activities, including, but not limited to: FGF activity; and an FGF-specific immunogenic activity. In accordance with the present invention, at least two novel classes of FGF have been identified, e.g., FGF-20 and FGF-23.

An “FGF activity” means, e.g., promoting wound healing; promoting neuronal survival; stimulating cell proliferation, e.g., proliferation of stem cells, fibroblasts, neurons, glia, oligodendrocytes, Schwann cells, or progenitors thereof; modulating differentiation of cells; inducing embryonic development; stimulating neurite outgrowth; enhancing recovery from nerve or neuronal damage; stimulating myelination; stimulating angiogenesis; receptor binding activity; modulating tumorogenesis, etc.

An “FGF-specific immunogenic activity” means, e.g., that an FGF polypeptide elicits an immunological response which is selective for FGF, e.g., an immunological response which is selective for mammalian FGF-20. Thus, the stimulation of antibodies, T-cells, macrophages, B-cells, dendritic cells, etc., by an amino acid sequence selected from a mammalian FGF, e.g., an FGF in FIGS. 1 and 2, is a specific immunogenic activity. These responses can be measured routinely.

FGF, such as FGF-20 or -23, is a full-length mammalian polypeptide having an amino acid sequence which is obtainable from a natural source and which has one or more of the aforementioned activities. It can have sequences as shown in FIGS. 1 and 2, having an open-reading frame that begins with an initiation codon and ends with a stop codon. It includes naturally-occurring normal, naturally-occurring mutant, and naturally-occurring polymorphic, including single nucleotide polymorphisms (SNP), etc., sequences. Natural sources include, e.g., living cells, e.g., obtained from tissues or whole organisms, cultured cell lines, including primary and immortalized cell lines, biopsied tissues, etc.

The present invention also relates to fragments of a mammalian FGF. The fragments are preferably “biologically active.” By “biologically active,” it is meant that the polypeptide fragment possesses an activity in a living system or with components of a living system. Biological activities include those mentioned, e.g., FGF-activity, such as FGF-receptor binding activity, and FGF-immunogenic activity. Fragments can be prepared according to any desired method, including, chemical synthesis, genetic engineering, cleavage products, etc. A biological-fragment of an FGF includes polypeptides which have had amino acid sequences removed or modified at either the carboxy- or amino-terminus of the protein.

Any pubicly available nucleic acid fragments and polypeptide fragments of FGF-20 and FGF-23, or homologous fragments thereof, are excluded from the present invention, e.g., g5762262 which is similar sequence identified from Xenopus laevis. The nucleotide and amino acid sequences of publicly available nucleic acids can be identified by searching publicly available databases.

The present invention also relates to a FGF-20 having a deduced sequence of amino acids 1 to 211 as shown in FIG. 1, and a FGF-23 having a deduced sequence of amino acids 1 to 169 as shown in FIG. 2. FGF-20 has predicted molecular weight of about 23.5 kdal and a predicted pI of about 9.25. FGF-23 has predicted molecular weight of about 19.6 kdal and a predicted pI of about 12.32.

For proteins degree of identity means number of identical amino acids/total number of amino acid residues in the protein: Degree of similarity means (number of identical amino acid residues plus number of conservatively substituted amino acids (like V for L, etc)/total number of amino acid residues. For DNA identity is the same as similarity and means the number of identical nucleotides/total length.

A FGF polypeptide of the invention, e.g., having an amino acid sequence as shown in FIGS. 1 and 2, can be analyzed by any suitable methods to identify other structural and/or functional domains in the polypeptide, including membrane spanning regions, hydrophobic regions. For example, an FGF polypeptide can be analyzed by methods disclosed in, e.g., Kyte and Doolittle, J. Mol. Bio., 157:105, 1982; EMBL Protein Predict; Rost and Sander, Proteins, 19:55-72, 1994.

Other homologs of FGFs of the present invention can be obtained from mammalian and non-mammalian sources according to various methods. For example, hybridization with oligonucleotides derive from FIGS. 1 and 2 can be employed to select homologs, e.g., as described in Sambrook et al., Molecular Cloning, Chapter 11, 1989. Such homologs can have varying amounts of nucleotide and amino acid sequence identity and similarity to GENE. Mammalian organisms include, e.g., rodents, mouse, rats, hamsters, monkeys, pigs, cows, etc. Non-mammalian organisms include, e.g., vertebrates, invertebrates, zebra fish, chicken, Drosophila, C. elegans, Xenopus, yeast such as S. pombe, S. cerevisiae, roundworms, prokaryotes, plants, Arabidopsis, viruses, artemia, etc.

The invention also relates to FGF-specific amino acid sequences, e.g., a defined amino acid sequence which is found in the particular sequences of FIGS. 1 and 2, conserved amino acid motifs found in the FGFs of the present invention. Comparisons between related proteins, such as other related FGFs (see, e.g., Venkataraman et al., Proc. Natl. Acad. Sci., 96:3658-3663, 1999), can be used to select sequences specific for FGFs.

For example, protein sequences of FGF-20 and -23 were aligned, and amino acid motifs were generated based on the conserved areas of homology a shown in FIGS. 1 and 2. The present invention relates to any nucleic acid or polypeptide sequences thereof, e.g., polypeptides which comprises three or more conserved or homologous residues, such as, e.g., LYGS, HFLP, VQGTR, RIEENGHNTY, QFEENWYNTY, AGTPSA, AAERSA, etc. Other specific and/or conserved amino acid sequences can be found routinely, e.g., by searching a gene/protein database using the BLAST set of computer programs. An FGF-specific amino acid sequence or motif can be useful to produce peptides as antigens to generate an immune response specific for it. Antibodies obtained by such immunization can be used as a specific probe for a mammalian FGF protein for diagnostic or research purposes.

As mentioned, polypeptides of the present invention can comprise various amino acid sequences for an FGF (e.g., a full-length sequence, i.e., having a start and stop codon as shown in FIGS. 1 and 2, a mature amino acid sequence (i.e., where the FGF polypeptide is produced as a precursor which is processed into a mature polypeptide, or fragments thereof). Useful fragments include, e.g., fragments comprising, or consisting essentially of, any of the aforementioned domains and specific and conserved amino acid sequences.

A fragment of an FGF polypeptide of the present invention can be selected to have a specific biological activity, e.g., FGF receptor binding activity or immunogenic activity.

The measurement of these activities is described below and in the examples. These peptides can also be identified and prepared as described in EP 496 162. A useful fragment can comprise, or consist essentially of, e.g., about nine contiguous amino acids, preferably about 10, 15, 20, 30, 40, etc. contiguous amino acids of FIGS. 1 and 2.

A polypeptide of the present invention can also have 100% or less amino acid sequence identity to the amino acid sequence set forth in FIGS. 1 and 2. For the purposes of the following discussion: Sequence identity means that the same nucleotide or amino acid which is found in the sequence set forth in FIGS. 1 and 2 is found at the corresponding position of the compared sequence(s). A polypeptide having less than 100% sequence identity to the amino acid sequences set forth in FIGS. 1 and 2 can contain various substitutions from the naturally-occurring sequence, including homologous and non-homologous amino acid substitutions. See below for examples of homologous amino acid substitution. The sum of the identical and homologous residues divided by the total number of residues in the sequence over which the FGF polypeptide is compared is equal to the percent sequence similarity. For purposes of calculating sequence identity and similarity, the compared sequences can be aligned and calculated according to any desired method, algorithm, computer program, etc., including, e.g., FASTA, BLAST. A polypeptide having less than 100% amino acid sequence identity to the amino acid sequence of FIGS. 1 and 2 can have about 99%, 98%, 97%, 95%, 90.5%, 90%, 85%, 70%, or as low as about 60% sequence identity.

The present invention also relates to FGF polypeptide muteins of FGF-21 and -23, i.e., any polypeptide which has an amino acid sequence which differs in amino acid sequence from an amino acid sequence obtainable from a natural source (a fragment of a mammalian FGF does not differ in amino acid sequence from a naturally-occurring FGF although it differs in amino acid number). Thus, FGF polypeptide muteins comprise amino acid substitutions, insertions, and deletions, including non-naturally occurring amino acids.

Muteins to an FGF amino acid sequence of the invention can also be prepared based on homology searching from gene data banks, e.g., Genbank, EMBL. Sequence homology searching can be accomplished using various methods, including algorithms described in the BLAST family of computer programs, the Smith-Waterman algorithm, etc. A mutein(s) can be introduced into a sequence by identifying and aligning amino acids within a domain which are identical and/or homologous between polypeptides and then modifying an amino acid based on such alignment. For instance, FGF of the present invention shares sequence identity with various known FGFs, e.g., Venkataraman et al., Proc. Natl. Acad. Sci., 96:3658-3663, 1999. Alignments between these polypeptides, especially at the conserved amino acid residues identified in Table 1 of Venkataraman et al. amino acid substitutions, can identify residues whose modification would be expected to reduce, decrease, or, eliminate a biological activity of an FGF, such as a receptor binding activity, etc. For instance, where alignment reveals identical amino acids conserved between two or more domains, elimination or substitution of the amino acid(s) would be expected to adversely affect its biological activity.

Amino acid substitution can be made by replacing one homologous amino acid for another. Homologous amino acids can be defined based on the size of the side chain and degree of polarization, including, small nonpolar: cysteine, proline, alanine, threonine; small polar: serine, glycine, aspartate, asparagine; large polar: glutamate, glutamine, lysine, arginine; intermediate polarity: tyrosine, histidine, tryptophan; large nonpolar: phenylalanine, methionine, leucine, isoleucine, valine. Homologous acids can also be grouped as follows: uncharged polar R groups, glycine, serine, threonine, cysteine, tyrosine, asparagine, glutamine; acidic amino acids (negatively charged), aspartic acid and glutamic acid; basic amino acids (positively charged), lysine, arginine, histidine. Homologous amino acids also include those described by Dayhoff in the Atlas of Protein Sequence and Structure 5, 1978, and by Argos in EMBO J., 8, 779-785, 1989.

The invention relates to mutein polypeptides and mutein nucleic acids coding for such polypeptides. Thus, the present invention relates to nucleotide sequences of FIGS. 1 and 2, wherein said nucleic acids code for a polypeptide and one or more amino acid positions are substituted or deleted, or both, and the polypeptide coded for by the nucleic acid has a biological activity, such as enhancing recovery from nerve or neuronal damage. A polypeptide mutein, and its corresponding nucleotide coding sequence, can have an amino acid sequence as set forth in FIGS. 1 and 2 except where one or more positions are substituted by homologous amino acids, e.g., where there are 1, 5, 10, 15, or 20 substitutions. How a modification affects the mentioned activities can be measured according to the methods described above, below, and as the skilled worker in the field would know. For example, various methods of assaying FGF activity are known in the art, including, e.g., assays that measure neuronal survival and other neutrotropic activities, such as the ones described in the examples and in Kanda et al., Int. J. Devl. Neuroscience, 12(3): 191-200, 1999, and FGF-receptor binding assays.

As mentioned, amino acid substitutions can also be made based on analogy to related other FGFs. Other mutations could be selected routinely by modifying or mutating a nucleotide sequence of FIGS. 1 and 2, and selecting for those mutations that affect one or more its activities, e.g., by measuring activity according to the methods and examples described below.

A mammalian FGF of the present invention, fragments, or substituted polypeptides thereof, can also comprise various modifications, where such modifications include lipid modification, methylation, phosphorylation, glycosylation, covalent modifications (e.g., of an R-group of an amino acid), amino acid substitution, amino acid deletion, or amino acid addition. Modifications to the polypeptide can be accomplished according to various methods, including recombinant, synthetic, chemical, etc.

Polypeptides of the present invention (e.g., full-length, fragments thereof, mutations thereof) can be used in various ways, e.g., in assays, as immunogens for antibodies as described below, as biologically-active agents (e.g., having one or more of the activities associated with an FGF of the present invention).

A polypeptide coding for an FGF of the present invention, a derivative thereof, or a fragment thereof, can be combined with one or more structural domains, functional domains, detectable domains, antigenic domains, and/or a desired polypeptide of interest, in an arrangement which does not occur in nature, i.e., not naturally-occurring. A polypeptide comprising such features is a chimeric or fusion polypeptide. Such a chimeric polypeptide can be prepared according to various methods, including, chemical, synthetic, quasi-synthetic, and/or recombinant methods. A chimeric nucleic acid coding for a chimeric polypeptide can contain the various domains or desired polypeptides in a continuous (e.g., with multiple N-terminal domains to stabilize or enhance activity) or interrupted open reading frame, e.g., containing introns, splice sites, enhancers, etc. The chimeric nucleic acid can be produced according to various methods. See, e.g., U.S. Pat. No. 5,439,819. A domain or desired polypeptide can possess any desired property, including, a biological function such as signaling, growth promoting, cellular targeting (e.g., signal sequence, targeting sequence, such as targeting to the endoplasmic reticulum or nucleus), etc., a structural function such as hydrophobic, hydrophilic, membrane-spanning, etc., receptor-ligand functions, and/or detectable functions, e.g., combined with enzyme, fluorescent polypeptide, green fluorescent protein, (Chalfie et al., Science, 263:802, 1994; Cheng et al., Nature Biotechnology, 14:606, 1996; Levy et al., Nature Biotechnology, 14:610, 1996), etc. In addition, a polypeptide, or a part of it, can be used as a selectable marker when introduced into a host cell. For example, a nucleic acid coding for an amino acid sequence according to the present invention can be fused in-frame to a desired coding sequence and act as a tag for purification, selection, or marking purposes. The region of fusion can encode a cleavage site to facilitate expression, isolation, purification, etc.

A polypeptide according to the present invention can be produced in an expression system, e.g., in vivo, in vitro, cell-free, recombinant, cell fusion, etc., according to the present invention. Modifications to the polypeptide imparted by such systems include glycosylation, amino acid substitution (e.g., by differing codon usage), polypeptide processing such as digestion, cleavage, endopeptidase or exopeptidase activity, attachment of chemical moieties, including lipids and phosphates, etc.

A polypeptide according to the present invention can be recovered from natural sources, transformed host cells (culture medium or cells) according to the usual methods, including, detergent extraction (e.g., non-ionic detergent, Triton X-100, CHAPS, octylglucoside, Igepal CA-630), ammonium sulfate or ethanol precipitation, acid extraction, anion or cation exchange chromatography, phosphocellulose chromatography, hydrophobic interaction chromatography, hydroxyapatite chromatography, lectin chromatography, gel electrophoresis. Protein refolding steps can be used, as necessary, in completing the configuration of the mature protein. Finally, high performance liquid chromatography (HPLC) can be employed for purification steps. An FGF polypeptide can also be isolated as described for other FGF proteins as the skilled worker would know, e.g., as described in the following which describe the isolation of various FGFs, U.S. Pat. Nos. 5,604,293, 5,395,756, 5,155,214, 4,902,782, and Santos-Ocampo et al., J. Biol. Chem., 271:1726-1731, 1996 (purifying FGF from a bacterial host, such as E. coli). Another approach is express FGF recombinantly with an affinity tag (Flag epitope, HA epitope, myc epitope, 6× His, maltose binding protein, chitinase, etc) and then purify by anti-tag antibody-conjugated affinity chromatography.

The present invention also relates to nucleic acids, such as DNAs and RNAs coding for the FGF polypeptides, and fragments thereof, of the present invention. An FGF nucleic acid (such as FGF-20 or -23), or fragment thereof, is a nucleic acid having a nucleotide sequence obtainable from a natural source. It therefore includes naturally-occurring, normal, naturally-occurring mutant, and naturally-occurring polymorphic alleles (e.g., SNPs), etc. Natural sources include, e.g., living cells obtained from tissues and whole organisms, tumors, cultured cell lines, including primary and immortalized cell lines.

A nucleic acid sequence of the invention can contain the complete coding sequence as shown in FIGS. 1 and 2, degenerate sequences thereof, and fragments thereof. A nucleic acid according to the present invention can also comprise a nucleotide sequence which is 100% complementary, e.g., an anti-sense, to any nucleotide sequence mentioned above and below.

A nucleic acid according to the present invention can be obtained from a variety of different sources. It can be obtained from DNA or RNA, such as polyadenylated mRNA, e.g., isolated from tissues, cells, or whole organism. The nucleic acid can be obtained directly from DNA or RNA, or from a cDNA library. The nucleic acid can be obtained from a cell or tissue (e.g., from an embryonic or adult heart or skeletal cells or tissues) at a particular stage of development, having a desired genotype, phenotype etc.

As described for the FGF polypeptide described above, a nucleic acid comprising a nucleotide sequence coding for a polypeptide according to the present invention can include only coding sequence; a coding sequence and additional coding sequence (e.g., sequences coding for leader, secretory, targeting, enzymatic, fluorescent or other diagnostic peptides), coding sequences and non-coding sequences, e.g., untranslated sequences at either a 5′ or 3′ end, or dispersed in the coding sequence, e.g., introns. A nucleic acid comprising a nucleotide sequence coding without interruption for a polypeptide means that the nucleotide sequence contains an amino acid coding sequence for an FGF, with no non-coding nucleotides interrupting or intervening in the coding sequence, e.g., absent intron(s). Such a nucleotide sequence can also be described as contiguous. A genomic DNA coding for a human, mouse, or other mammalian FGF gene, etc., can be obtained routinely.

A nucleic acid according to the present invention also can comprise an expression control sequence operably linked to a nucleic acid as described above. The phrase “expression control sequence” means a nucleic acid sequence which regulates expression of a polypeptide coded for by a nucleic acid to which it is operably linked. Expression can be regulated at the level of the mRNA or polypeptide. Thus, the expression control sequence includes mRNA-related elements and protein-related elements. Such elements include promoters, enhancers (viral or cellular), ribosome binding sequences, transcriptional terminators, etc. An expression control sequence is operably linked to a nucleotide coding sequence when the expression control sequence is positioned in such a manner to effect or achieve expression of the coding sequence. For example, when a promoter is operably linked 5′ to a coding sequence, expression of the coding sequence is driven by the promoter. Expression control sequences can be heterologous or endogenous to the normal gene.

A nucleic acid in accordance with the present invention can be selected on the basis of nucleic acid hybridization. The ability of two single-stranded nucleic acid preparations to hybridize together is a measure of their nucleotide sequence complementarity, e.g., base-pairing between nucleotides, such as A-T, G-C, etc. The invention thus also relates to nucleic acids, and their complements, which hybridize to a nucleic acid comprising a nucleotide sequence as set forth in FIGS. 1 and 2. A nucleotide sequence hybridizing to the latter sequence will have a complementary nucleic acid strand, or act as a template for one in the presence of a polymerase (i.e., an appropriate nucleic acid synthesizing enzyme). The present invention includes both strands of nucleic acid, e.g., a sense strand and an anti-sense strand.

Hybridization conditions can be chosen to select nucleic acids which have a desired amount of nucleotide complementarity with the nucleotide sequence set forth in FIGS. 1 and 2. A nucleic acid capable of hybridizing to such sequence, preferably, possesses, e.g., about 85%, more preferably, 90%, 92%, and even more preferably, 95%, 97%, or 100% complementarity, between the sequences. The present invention particularly relates to nucleic acid sequences which hybridize to the nucleotide sequence set forth in FIGS. 1 and 2 under low or high stringency conditions.

Nucleic acids which hybridize to FGF sequences can be selected in various ways. For instance, blots (i.e., matrices containing nucleic acid), chip arrays, and other matrices comprising nucleic acids of interest, can be incubated in a prehybridization solution (6×SSC, 0.5% SDS, 100 μg/ml denatured salmon sperm DNA, 5× Denhardt's solution, and 50% formamide), at 30° C., overnight, and then hybridized with a detectable oligonucleotides probe, (see below) in a hybridization solution (e.g., 6×SSC, 0.5% SDS, 100 μg/ml denatured salmon sperm DNA and 50% formamide), at 42° C., overnight in accordance with known procedures. Blots can be washed at high stringency conditions that allow, e.g., for less than 5% bp mismatch (e.g., wash twice in 0.1% SSC and 0.1% SDS for 30 min at 65° C.), i.e., selecting sequences having 95% or greater sequence identity. Other non-limiting examples of high stringency conditions includes a final wash at 65° C. in aqueous buffer containing 30 mN NaCl and 0.5% SDS. Another example of high stringent conditions is hybridization in 7% SDS, 0.5 M NaPO4, pH 7, 1 mM EDTA at 50° C., e.g., overnight, followed by one or more washes with a 1% SDS solution at 42° C.

Whereas high stringency washes can allow for less than 5% mismatch, relaxed or low stringency wash conditions (e.g., wash twice in 0.2% SSC and 0.5% SDS for 30 min at 37° C.) can permit up to 20% mismatch. Another non-limiting example of low stringency conditions includes a final wash at 42° C. in a buffer containing 30 mM NaCl and 0.5% SDS. Washing and hybridization can also be performed as described in Sambrook et al., Molecular Cloning, 1989, Chapter 9.

Hybridization can also be based on a calculation of melting temperature (Tm) of the hybrid formed between the probe and its target, as described in Sambrook et al. Generally, the temperature Tm at which a short oligonucleotide (containing 18 nucleotides or fewer) will melt from its target sequence is given by the following equation: Tm=(number of A's and T's)×2° C.+(number of C's and G's)×4° C. For longer molecules, Tm=81.5+16.6 log 10[Na+]+0.41(% GC)−600/N where [Na+] is the molar concentration of sodium ions, % GC is the percentage of GC base pairs in the probe, and N is the length. Hybridization can be carried out at several degrees below this temperature to ensure that the probe and target can hybridize. Mismatches can be allowed for by lowering the temperature even further.

Stringent conditions can be selected to isolate sequences, and their complements, which have, e.g., at least about 95%, preferably 97%, nucleotide complementarity between the probe (e.g., an oligonucleotide of an FGF and target nucleic acid.

According to the present invention, a nucleic acid or polypeptide can comprise one or more differences in the nucleotide or amino acid sequence set forth in FIGS. 1 and 2. Changes or modifications to the nucleotide and/or amino acid sequence can be accomplished by any method available, including directed or random mutagenesis.

A nucleic acid coding for a mammalian FGF, such as FGF-20 or -23, according to the invention can comprise nucleotides which occur in a naturally-occurring gene e.g., naturally-occurring polymorphisms, normal or mutant alleles (nucleotide or amino acid), mutations which are discovered in a natural population of mammals, such as humans, monkeys, pigs, mice, rats, or rabbits. For example, a human FGF nucleic acid or polypeptide comprises nucleotides or amino acids which occur in a naturally-occurring human population. By the term naturally-occurring, it is meant that the nucleic acid is obtainable from a natural source, e.g., animal tissue and cells, body fluids, tissue culture cells, forensic samples. Naturally-occurring mutations can include deletions (e.g., a truncated amino- or carboxy-terminus), substitutions, inversions, or additions of nucleotide sequence. These genes can be detected and isolated by nucleic acid hybridization according to methods which one skilled in the art would know. A nucleotide sequence coding for a mammalian FGF of the invention can contain codons found in a naturally-occurring gene, transcript, or cDNA, for example, e.g., as set forth in FIGS. 1 and 2, or it can contain degenerate codons coding for the same amino acid sequences. For instance, it may be desirable to change the codons in the sequence to optimize the sequence for expression in a desired host.

A nucleic acid according to the present invention can comprise, e.g., DNA, RNA, synthetic nucleic acid, peptide nucleic acid, modified nucleotides, or mixtures. A DNA can be double- or single-stranded. Nucleotides comprising a nucleic acid can be joined via various known linkages, e.g., ester, sulfamate, sulfamide, phosphorothioate, phosphoramidate, methylphosphonate, carbamate, etc., depending on the desired purpose, e.g., resistance to nucleases, such as RNAase H, improved in vivo stability, etc. See, e.g., U.S. Pat. No. 5,378,825.

Various modifications can be made to the nucleic acids, such as attaching detectable markers (avidin, biotin, radioactive elements), moieties which improve hybridization, detection, or stability. The nucleic acids can also be attached to solid supports, e.g., nitrocellulose, magnetic or paramagnetic microspheres (e.g., as described in U.S. Pat. No. 5,411,863; U.S. Pat. No. 5,543,289; for instance, comprising ferromagnetic, supermagnetic, paramagnetic, superparamagnetic, iron oxide and polysaccharide), nylon, agarose, diazotized cellulose, latex solid microspheres, polyacrylamides, etc., according to a desired method. See, e.g., U.S. Pat. Nos. 5,470,967; 5,476,925; 5,478,893.

Another aspect of the present invention relates to oligonucleotides or nucleic acid probes. Such oligonucleotides or nucleic acid probes can be used, e.g., to detect, quantitate, or isolate a mammalian FGF nucleic acid in a test sample, or to identify FGF homologs. In a preferred embodiment, the nucleic acids can be utilized as oligonucleotide probes, e.g., in PCR, differential display, gene chips (e.g., Affymetrix GeneChips; U.S. Pat. No. 5,143,854, U.S. Pat. No, 5,424,186; U.S. Pat. No. 5,874,219; PCT WO 92/10092; PCT WO 90/15070), and other available methods. Detection can be desirable for a variety of different purposes, including research, diagnostic, and forensic. For diagnostic purposes, it may be desirable to identify the presence or quantity of a nucleic acid sequence in a sample, where the sample is obtained from tissue, cells, body fluids, etc. In a preferred method, the present invention relates to a method of detecting a nucleic acid comprising, contacting a target nucleic acid in a test sample with an oligonucleotide under conditions effective to achieve hybridization between the target and oligonucleotide; and detecting hybridization. An oligonucleotide in accordance with the invention can also be used in synthetic nucleic acid amplification such as PCR (e.g., Saiki et al., Science, 241:53, 1988; U.S. Pat. No. 4,683,202; PCR Protocols: A Guide to Methods and Applications, Innis et al., eds., Academic Press, New York, 1990); differential display (See, e.g., Liang et al., Nucl. Acid. Res., 21:3269-3275, 1993; U.S. Pat. No. 5,599,672; WO97/18454).

Detection can be accomplished in combination with oligonucleotides for other genes, e.g., genes involved in signal transduction, growth, cancer, apoptosis, or any of the genes mentioned above or below, etc. Oligonucleotides can also be used to test for mutations, e.g., using mismatch DNA repair technology as described in U.S. Pat. No. 5,683,877; U.S. Pat. No. 5,656,430; Wu et al., Proc. Natl. Acad. Sci., 89:8779-8783, 1992.

Oligonucleotides of the present invention can comprise any continuous nucleotide sequence of FIGS. 1 and 2 or a complement thereto, or any of the sequences, or complements thereto. These oligonucleotides (nucleic acid) according to the present invention can be of any desired size, e.g., about 10-200 nucleotides, 12-100, preferably 12-50, 12-25, 14-16, at least about 15, at least about 20, at least about 25, etc. The oligonucleotides can have non-naturally-occurring nucleotides, e.g., inosine, AZT, 3TC, etc. The oligonucleotides can have 100% identity or complementarity to a sequence of FIGS. 1 and 2, or it can have mismatches or nucleotide substitutions, e.g., 1, 2, 3, 4, or 5 substitutions. For example, the oligonucleotides can have 70-99% identity, e.g., 90, 95 or 97% identity, to a sequence of FIG. 1 or 2. In accordance with the present invention, the oligonucleotide can comprise a kit, where the kit includes a desired buffer (e.g., phosphate, tris, etc.), detection compositions, etc. The oligonucleotide can be labeled or unlabeled, with radioactive or non-radioactive labels as known in the art.

Another aspect of the present invention is a nucleotide sequence which is unique to a mammalian FGF. By a unique sequence to an FGF, it is meant a defined order of nucleotides which occurs in FGF, e.g., in the nucleotide sequences of FIGS. 1 and 2, but rarely or infrequently in other nucleic acids, especially not in an animal nucleic acid, preferably mammal, such as human, rat, mouse, etc. Unique nucleotide sequences include the sequences, or complements thereto, coding for amino acids as shown in 1 and 2 and FIGS. 1 and 2. Such sequences can be used as probes in any of the methods described herein or incorporated by reference. Both sense and antisense nucleotide sequences are included. A unique nucleic acid according to the present invention can be determined routinely. A nucleic acid comprising such a unique sequence can be used as a hybridization probe to identify the presence of, e.g., human or mouse FGF, in a sample comprising a mixture of nucleic acids, e.g., on a Northern blot. Hybridization can be performed under high stringent conditions (see, above) to select nucleic acids (and their complements which can contain the coding sequence) having at least 95% identity (i.e., complementarity) to the probe, but less stringent conditions can also be used. A unique FGF nucleotide sequence can also be fused in-frame, at either its 5′ or 3′ end, to various nucleotide sequences as mentioned throughout the patent, including coding sequences for other parts of FGF, enzymes, GFP, etc, expression control sequences, etc.

As already discussed, hybridization can be performed under different conditions, depending on the desired selectivity, e.g., as described in Sambrook et al., Molecular Cloning, 1989. For example, to specifically detect FGF of the present invention, an oligonucleotide can be hybridized to a target nucleic acid under conditions in which the oligonucleotide only hybridizes to it, e.g., where the oligonucleotide is 100% complementary to the target. Different conditions can be used if it is desired to select target nucleic acids which have less than 100% nucleotide complementarity, at least about, e.g., 99%, 97%, 95%, 90%, 86.4%, 85%, 70%, 67%.

The nucleic acid according to the present invention can be labeled according to any desired method. The nucleic acid can be labeled using radioactive tracers such as ³²P, ³⁵S, ¹²⁵I, ³H, or ¹⁴C, to mention some commonly used tracers. The radioactive labeling can be carried out according to any method such as, for example, terminal labeling at the 3′ or 5′ end using a radiolabeled nucleotide, polynucleotide kinase (with or without dephosphorylation with a phosphatase) or a ligase (depending on the end to be labeled). A non-radioactive labeling can also be used, combining a nucleic acid of the present invention with residues having immunological properties (antigens, haptens), a specific affinity for certain reagents (ligands), properties enabling detectable enzyme reactions to be completed (enzymes or coenzymes, enzyme substrates, or other substances involved in an enzymatic reaction), or characteristic physical properties, such as fluorescence or the emission or absorption of light at a desired wavelength, etc.

A nucleic acid according to the present invention, including oligonucleotides, anti-sense nucleic acid, etc., can be used to detect expression of FGF in whole organs, tissues, cells, etc., by various techniques, including Northern blot, PCR, in situ hybridization, differential display, nucleic acid arrays, dot blots, etc. Such nucleic acids can be particularly useful to detect disturbed expression, e.g., cell-specific and/or subcellular alterations, of FGF. The levels of FGF can be determined alone or in combination with other gene products, especially other gene products involved in neuronal pphysiology.

A nucleic acid according to the present invention can be expressed in a variety of different systems, in vitro and in vivo, according to the desired purpose. For example, a nucleic acid can be inserted into an expression vector, introduced into a desired host, and cultured under conditions effective to achieve expression of a polypeptide coded for by the nucleic acid. Effective conditions include any culture conditions which are suitable for achieving production of the polypeptide by the host cell, including effective temperatures, pH, medium, additives to the media in which the host cell is cultured (e.g., additives which amplify or induce expression such as butyrate, or methotrexate if the coding nucleic acid is adjacent to a dhfr gene), cycloheximide, cell densities, culture dishes, etc. A nucleic acid can be introduced into the cell by any effective method including, e.g., naked DNA, calcium phosphate precipitation, electroporation, injection, DEAE-Dextran mediated transfection, fusion with liposomes, association with agents which enhance its uptake into cells, viral transfection. A cell into which a nucleic acid of the present invention has been introduced is a transformed host cell. The nucleic acid can be extrachromosomal or integrated into a chromosome(s) of the host cell. It can be stable or transient. An expression vector is selected for its compatibility with the host cell. Host cells include, mammalian cells, e.g., COS, CV1, BHK, CHO, HeLa, LTK, NIH 3T3, 293, PAE, human, human fibroblast, human primary tumor cells, testes, glia, neurons, oligodendrocytes,glia, neuroblastoma, glioma, etc., insect cells, such as Sf9 (S. frugipeda) and Drosophila, bacteria, such as E. coli, Streptococcus, bacillus, yeast, such as Sacharomyces, S. cerevisiae, fungal cells, plant cells, embryonic stem cells (e.g., mammalian, such as mouse or human), neuronal stem cells, fibroblasts, muscle cells, cardiac cells, and T-cells.

Expression control sequences are similarly selected for host compatibility and a desired purpose, e.g., high copy number, high amounts, induction, amplification, controlled expression. Other sequences which can be employed include enhancers such as from SV40, CMV, RSV, inducible promoters, cell-type specific elements, or sequences which allow selective or specific cell expression. Promoters that can be used to drive its expression, include, e.g., the endogenous promoter, promoters of other genes in the cell signal transduction pathway, MMTV, SV40, trp, lac, tac, or T7 promoters for bacterial hosts; or alpha factor, alcohol oxidase, or PGH promoters for yeast. RNA promoters can be used to produced RNA transcripts, such as T7 or SP6. See, e.g., Melton et al., Nucleic Acid Res., 12(18):7035-7056, 1984; Dunn and Studier. J. Mol. Bio., 166:477-435, 1984; U.S. Pat. No. 5,891,636; Studier et al., Gene Expression Technology, Methods in Enzymology, 85:60-89, 1987.

A nucleic acid or polypeptide of the present invention can be used as a size marker in nucleic acid or protein electrophoresis, chromatography, etc. Defined restriction fragments can be determined by scanning the sequence for restriction sites, calculating the size, and performing the corresponding restriction digest.

An FGF polypeptide and nucleic acid of the present invention can be “isolated.” By the term “isolated,” it is meant that it is in a form in which it is not found in its original environment or in nature, e.g., more concentrated, more purified, separated from components, present in a lysate of a cell in which a heterologous FGF gene is expressed. When FGF is expressed as a heterologous gene in a transfected cell line, a gene in accordance with the present invention is introduced into a cell as described above, under conditions in which the gene is expressed. The term “heterologous” means that the gene has been introduced into the cell line by the “hand-of-man.” Introduction of a gene into a cell line is discussed above. The transfected (or transformed) cell expressing the FGF gene can be lysed as described in the examples and used in the method as a lysate (i.e., “isolated”) or the cell line can be used intact.

Generally, the term “effective conditions” means, e.g., a milieu in which the desired effect is achieved. Such a milieu, includes, e.g., buffers, oxidizing agents, reducing agents, pH, co-factors, temperature, ion concentrations, suitable age and/or stage of cell (such as, in particular part of the cell cycle, or at a particular stage where particular genes are being expressed) where cells are being used, culture conditions (including substrate, oxygen, carbon dioxide, etc.).

To enhance stability, the administered nucleic acid can be modified, e.g., to make it resistant to cellular enzymes, oxidation, reduction, nucleases, etc, or to enhance its uptake into cells. Any suitable modification can be used, including, e.g., phosporothioates, methylphosphonates, phosphodiester oligonucleotide linked to an acridine intercalating agent and/or a hydrophobic tail, psoralen derivatives, 2′-ribose modifications, pentose sugar derivatives, nitrogen base derivatives, etc. See, e.g., U.S. Pat. No. 5,576,208 and U.S. Pat. No. 5,744,362. See, above, for other derivatives, modifications, etc. which can be useful in the invention. In general, an antisense nucleic acid of the present invention can comprise monomers of naturally-occurring nucleotides, non-naturally-occurring nucleotides, and combinations thereof to enhance cellular uptake and/or stability.

Antisense can be administered as naked nucleic acid, complexed or encapsulated with and by other agents which facilitate its uptake into a cell, injected into cells, or any suitable delivery means.

The present invention also relates to methods of using an FGF of the present invention, such as FGF-20 and FGF-23. Such methods involve administering an effective amount of an FGF or a nucleic acid encoding the FGF of the present invention to a host for one or more the following purposes: promoting survival and/or proliferation of, e.g., neurons, oligodendrocytes, Schwann cells, stem cells, especially neural stem cells, endothelial cells, keratinocytes, and any cell type which is capable of responding to an FGF-20 or FGF-23, e.g., cells which express the cognate receptor (such as FGFR 1-4) on their cell surface, or progenitors thereof; promoting wound healing; modulating differentiation of cells; inducing embryonic development; stimulating neurite outgrowth; enhancing recovery from nerve or neuronal damage; stimulating myelination; stimulating angiogenesis; receptor binding activity.

The present invention also relates to the indications and methods of using the FGF of the present invention, such as FGF-20, and FGF-23, or a nucleic acid encoding the FGF. Such methods involve administering an effective amount of FGF of a present invention to a host for one or more of the following purposes: enhancing recovery from nerve and axonal damage; stimulating myelination, angiogenesis, wound healing, ulcer healing, inducing repair of a bone defect, promoting graft survival and inducing embryonic development. The above mentioned applications would be a result of a potential FGF activity promoting cell survival and/or proliferation, inhibiting and/or stimulating differentiation of certain cell types. FGF can induce cell survival/proliferation of stem cells, progenitors, precursors and mature cells of the following origin: neurons, oligodendrocytes, Schwann cells, endothelial cells keratinocytes and other cell types expressing any of the FGF receptors. In addition, FGFs can induce differentiation of neuronal progenitors by inducing neurite outgrowth/extension.

The following in vitro and in vivo assays can be performed in order to measure the activity of FGFs on the above-described cell functions:

In vitro ASSAYS:

Induction of oligodendrocyte proliferation in vitro: Oligodendrocytes used for measuring the effects of GF on cell proliferation are either established cell lines such as N 20.1 or primary rodent oligodendrocytes. Primary rodent (rat) oligodendrocytes and oligodendrocyte progenitors can be isolated and purified by either one of the following techniques: differential adhesion technique (Mitrovic et al., 1994); Percol gradient centrifugation (Mattera et al., Neurochem. Int. 1984, 6(1) 41-50 and Kim et al., J Neurol Sci December 1983:62(1-3):295-301) and immunoseparation. Regardless of the source of oligodendroycte cells (primary cells or cell line) or their method of isolation and purification, the oligodendrocyte proliferation assay can be carried out for time periods of 3, 5 and 7 days. Positive controls are other members of FGF family such as FGF-2 or FGF-9. Cell proliferation is measured as MTT assay and ³H-Thymidine incorporation assay. See also assays for oligodendrocyte proliferation in Danilenko, et al., Arch Biochem Biophys. Jan. 1, 1999:361(1): 34-46.

Induction of neurite outgrowth: PC 12 assays: Novel FGF family members can be tested for the induction of differentiation and neurite outgrowth in the PC-12 cell line (derived from a rat pheochromocytoma tumor) (Rydel, 1987 Greene, 1976). Additionally, since a portion of the NGF induced response has been shown to be due to the autocrine NGF-induced production of FGF-2, one can examine the effects of novel FGFs on the upregulation of NGF production by PC 12 cells (Chevet et al., J. Biol Chem. Jul. 23, 1999:274(3): 20901-8).

Neurite outgrowth in dorsal root ganglia (DRG): DRG are isolated by dissecting fetal rat DRG and culturing them in neurobasal media; the extent of neurite outgrowth in DRGs is assessed visually and quantified by determining the number and the length of neurites as compared with non-treated controls.

Assays can be performed on cells of fibroblast and endothelial origin. For fibroblasts, a modification of a NIH 3T3 proliferation assay can be used. For determining the effects of FGFs on the induction of endothelial cell proliferation, the following cells can be used: HUVEC cells, microvascular endothelial cells and aortic endothelial cells. An in vitro assay relevant for determining the therapeutic potential of FGFs as a potential therapeutic agent for the treatment of wounds, ulcers or bone damage can be performed as described in literature.

Other assays which correlate with CNS regeneration include assays of activation of growth- or survival-related gene expression (Meiners, et al., Dev Biol. December 1993: 160((2): 480-93), of modulation of other growth factors in vivo (Yoshida, 1992), of modulation of neuronal electrophysiology (Terlau, 1990), of activity as mitogens or differentation factors for oligodendrocytes, Schwan cells or astrocytes (Genburger, 1987; Stemple, 1988; Kalcheim, Dev Biol. July 1989:134(1):1-10; Murphy, 1990), of the promotion of in vitro survival of cortical, hippocampal, motor, sensory, sympathetic, or parasympathetic neurons (Eckstein, 1994; Unsicker, et al., Ann N.Y. Acad Sci. 1991:638:300-5; Grothe, et al., Int J Dev Biol. February 1996:40(1): 403-10), of the promotion of motor neuron survival in vitro, or the like.

In vivo ASSAYS:

Remyelinating potential of novel FGFs can be examined, e.g., in the following models: a) myelin defficient animal models such as transplantation of SVZ cells from donor animals treated with FGF, into myelin deficient mice and measurement of oligodendrocyte expansion in vivo; b) demyelinating animal models such as PLT induced CR-EAE and MBP adoptive transfer induced CR-EAE. See also assays described in Gumpel, 1992 and Hinks, et al., Mol Cell Neurosci. August 1999:14(2): 153-68.

FGFs can be tested for their ability to induce neuroregeneration—neuroprotection in the following in vivo models: mechanical damage/injury (transection of fimbria fornix pathway, sciatic nerve, spinal cord, optic nerve and transection of DRG); models of neuronal damage due to cerebrovascular insult such as carotid artery occlusion, temporary MCAO occlusion and hypoxic-ichemic cerebral insult; and in chemically induced neurodegeneration due to MPTP induced lesions or KA induced seizures.

Typical in vivo assays include, for example, measurement of reduction of neuronal loss after hippocampal ischemia (Sasaki, 1992; MacMillan, et al., Can J Neurol Sci February 1993:20(1): 37-40, promotion of the survival of cortical neurons following perforant path lesions (Gomez-Pinilla, 1992; Peterson, et al., J. Neurosci. Feb. 1, 1996:16(3): 886-98), protection of basal forebrain cholinergic neurons from injury induced degeneration and reduction of MPTP-induced or lesion-induced loss of substantia nigra neurons (Anderson, et al., Nature Mar. 24, 1998: 332(6162):360-1; Otto, 1989; Gomez-Pinilla, 1992; Otto, 1990); and long term grown of neural progenitor cells in vitro as “neurospheres” (reviewed in Svendsen, et al., Trends Neurosci. August 1999: 22(8): 357-64. See also the use of models for traumatic insult, such as optic nerve transection (Sievers, 1987); Sciatic nerve transection (Cordeiro, et al., Plast Reconstr Surg. June 1989:83(6): 1013-9; Khouri, et al., Microsurgery 1989:10(3): 206-9), transected DRG's (Aebischer, et al., J. Neurosci Res. Jul. 23, 1989 (3):282-9), spinal cord transection (Cheng, et al., Science Jul. 26, 1996:273 (5274): 510-3 1996) and facial nerve crush (Kuzis 1990); the use of models for cerebrovascular insult, such as hypoxemic-ischemic cerebral insult (MacMillen, 1993) and MCA occlusion (Kawamata, et al., Proc Natl Acad Sci U.S.A. Jul. 22, 1997:94(15): 8179-84; Schabitz, 1999); and other neurodegenerative models, such as kianic acid (KA) treatment (Liu, et al., Brain Res Oct. 29, 1993:626(1-2):335-8) or MND in wobbler mouse (Ikeda, et al., Neurol Res. December 1995: 17(6): 445-8).

By the term “administering,” it is meant that FGF, nucleic acid encoding the FGF, or other active agent, is delivered to the target, e.g., the injury, the damaged tissue, etc. FGF can be administered to any target (e.g., in vivo, in vitro, or in situ), including cells in culture and hosts having an injury, condition, or disease to be treated, by an effective route suitable to achieve an effect as described above, e.g., an FGF formulation can be administered by injection directly into, or close by, a target site. It can also be administered topically, enterally, parenterally, intravenously, intramuscularly, subcutaneously, orally, nasally, intracerebrally, intraventricularly, intracisternally, intracranially, implanted into desired location, e.g., in a gel foam, collagen filled nerve guide, etc., e.g., depending upon the location of the target site to be treated. FGF can also be administered continuously using an osmotic pump. An FGF can also be administered as a nucleic acid for uptake by cells. Methods to administer nucleic acid include those described above, and other conventional state-of-the-art techniques.

An effective amount of an FGF is administered to the target. Effective amounts are such amounts which are useful to achieve the desired effect, preferably a beneficial or therapeutic effect. Such amount can be determined routinely, e.g., by performing a dose-response experiment in which varying doses are administered to target cells to determine an effective amount in achieving the desired purpose, e.g., stimulating neurite outgrowth or promoting neuronal survival. Amounts are selected based on various factors, including the milieu to which the FGF is administered (e.g., a patient with a brain injury, animal model, tissue culture cells, etc.), the site of the cells to be treated, the age, health, gender, and weight of a patient or animal to be treated, etc.

In one aspect, the present invention relates to methods of treating neuronal injuries, such as nerve damage and trauma, spinal cord damage and trauma, damage to neuronal tissue produced by, e.g., ischemic attacks, infarction, hemorrhage, and aneurysm; treating a neuronal disease, e.g., neuronal degeneration diseases, such as Alzheimer's disease, Parkinson's disease, Huntington's disease, multiple sclerosis, myelopathy, myelitis, and syringomyelia, etc., comprising administering an effective amount of an FGF of the present invention.

The FGFs from this invention can be used for a treatment of neurodegenerative and demyelinating diseases of the CNS and PNS, characterized by the destruction of neurons and oligodendrocytes. FGF can be used as a remyelinating therapeutic for the treatment of Multiple Sclerosis and other primary and/or secondary demyelinating disease of CNS or PNS. Primary demyelinating diseases of CNS include adrenoleukodystrophies, leukoencephalopathies (such as progressive multifocal leukoencephalopathy), encephalomyelitis (like acute disseminated perivenous encaphalomyelitis). Secondary demyelination in CNS is represented as a formation of demyelinating lesions in CNS trauma, toxicity (cyanide, hexachlorphane) or ischemia (stroke). Demyelinating diseases of PNS include primary disorders like Guillian-Barre Syndrome (GBS), paraproteinemias, Chronic Inflammatory Demyelinating Polyneuropathy (CIDP). In addition, FGF will be used for the treatment of neurodegenerative diseases of CNS and PNS where neuronal damage is due to injury/trauma (mechanical, chemical, cerebrovascular insult and inflammation due to infection and autoimmune response) and for the treatment of other neurodegenerative diseases.

FGFs of the invention can also be used to promote graft survival. For example, FGF can be used to promote the survival of grafts (e.g., allogenic, isogenic or autologous) of a variety of cells, tissues or organs, such as skin, fascicles, tendons, bone, kidney, corneas, or the like. Transplants-of cells into the CNS or PNS of neuronal, glial or stem cell origin are also contemplated by the invention. Grafted material can be prepared from natural sources or by in vitro expansion of cells or tissue to be grafted or by using differentiated or non-differentiated stem cells. By the term “to promote” is meant herein to enhance the survival and/or proliferation of grafted cells, tissue or organs which have been treated with an FGF in comparison to cells, tissues or organs which are not so treated. Methods to assay for survival of grafts are conventional.

Assays for measuring graft survival are routine and well-known in the art. Conventional in vitro assays include, e.g., MTT, MTS, Thy incorporation, live/dead cell assays (e.g., double staining with calcein AM and ethidium homodimer-EthD-1), measurement of total cell number, e.g. by using microscopic evaluation or by physical methods of counting cells, such as using blood cell counters. Conventional in vivo methods include, e.g., for CNS indications, the detection of improved neurological function, or imaging techniques such as MTR, MRS, CT, or MRI, with or without Gd enhancement.

Other conditions which can be treated in accordance with the present invention include, prevention against myocardial damage due to MI, induction of angiogenesis, wound healing, ulcer healing, prevention of a bone destruction and induction of a new bone formation, promoting graft survival and inducing embryonic development.

FGF activities that would be useful in treating the above-described diseases/conditions include: promoting cell survival and/or proliferation, inhibiting and/or stimulating differentiation of the following cell types: induction of cell survival/proliferation of stem cells, progenitors, precursors and mature cells of the following origin: neurons, oligodendrocytes, Schwann cells, endothelial cells, keratinocytes, osteoblasts and other cell types expressing any of the FGF receptors. In addition, FGF effects on the induction of differentiation of neuronal progenitors by inducing neurite outgrowth/extension are considered useful in treating any kind of neuronal injury/damage.

By the term “treating,” is meant any effect that results in the improvement of the injury or disease, such as promoting survival of the neurons, glia, oligodendrocytes, astrocytes, Schwann cells, etc., stimulating neurite outgrowth, stimulating myelination, stimulating proliferation of cells, etc., as mentioned above. To treat such injuries and diseases, the FGF can be formulated as a composition, or nucleic acid, and applied to the injured or diseased area, e.g., using surgical techniques.

FGFs of the invention can also be administered for any of the treatment methods disclosed herein by the administration of nucleic acid, e.g., in methods of gene therapy. The gene delivery vehicle may be of viral or non-viral origin (see generally, Jolly, Cancer Gene Therapy 1:51-64 (1994) Kimura, Human Gene Therapy 5:845-852 (1994); Connelly, Human Gene Therapy 1:185-193 (1995); and Kaplitt, Nature Genetics 6:148-153 (1994). Gene therapy vehicles for delivery of constructs including a coding sequence of a therapeutic of the invention can be administered either locally or systemically. These constructs can utilize viral or non-viral vector approaches. Expression of such coding sequences can be induced using endogenous mammalian or heterologous promoters. Expression of the coding sequence can be either constitutive or regulated.

The present invention can employ recombinant retroviruses which are constructed to carry or express a selected nucleic acid molecule of interest. Retrovirus vectors that can be employed include those described in EP 0 415 731; WO 90/07936; WO 94/03622; WO 93/25698; WO 93/25234; U.S. Pat. No. 5,219,740; WO 93/11230; WO 93/10218; Vile and Hart, Cancer Res. 53:3860-3864 (1993); Vile and Hart, Cancer Res. 53:962-967 (1993); Ram et al., Cancer Res. 53:83-88 (1993); Takamiya et al., J. Neurosci. Res. 33:493-503 (1992); Baba et al., J. Neurosurg. 79:729-735 (1993); U.S. Pat. No. 4,777,127; GB Patent No. 2,200,651; and EP 0 345 242. Preferred recombinant retroviruses include those described in WO 91/02805.

Packaging cell lines suitable for use with the above-described retroviral vector constructs may be readily prepared (see PCT publications WO 95/30763 and WO 92/05266), and used to create producer cell lines (also termed vector cell lines) for the production of recombinant vector particles. Within particularly preferred embodiments of the invention, packaging cell lines are made from human (such as HT1080 cells) or mink parent cell lines, thereby allowing production of recombinant retroviruses that can survive inactivation in human serum.

The present invention also employs aphavirus-based vectors that can function as gene delivery vehicles. Such vectors can be constructed from a wide variety of alphaviruses, including, for example, Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250 ATCC VR-1249; ATCC VR-532). Representative examples of such vector systems include those described in U.S. Pat. Nos. 5,091,309; 5,217,879; and 5,185,440; and PCT Publication Nos. WO 92/10578; WO 94/21792; WO 95/27069; WO 95/27044; and WO 95/07994.

Gene delivery vehicles of the present invention can also employ parvovirus such as adeno-associated virus (AAV) vectors. Representative examples include the AAV vectors disclosed by Srivastava in WO 93/09239, Samulski et al., J Vir. 63:3822-3828 (1989); Mendelson et al., Virol. 166:154-165 (1988); and Flotte et al., P.N.A.S. 90:10613-10617 (1993).

Representative examples of adenoviral vectors include those described by Berkner, Biotechniques 6:616-627 (1988); Rosenfeld et al., Science 252:431-434 (1991); WO 93/19191; Kolls et al., P.N.A.S. 215-219 (1994); Kass-Eisler et al., P.N.A.S. 90:11498-11502 (1993); Guzman et al., Circulation 88:2838-2848 (1993); Guzman et al., Cir. Res. 73:1202-1207 (1993); Zabner et al., Cell 75:207-216 (1993); Li et al., Hum. Gene Ther. 4:403-409 (1993); Cailaud et al., Eur. J. Neurosci. 5: 1287-1291 (1993); Vincent et al., Nat. Genet. 5:130-134 (1993); Jaffe et al., Nat. Genet. 1:372-378 (1992); and Levrero et al., Gene 101:195-202 (1992). Exemplary adenoviral gene therapy vectors employable in this invention also include those described in WO 94/12649, WO 93/03769; WO 93/19191; WO 94/28938; WO 95/11984 and WO 95/00655. Administration of DNA linked to killed adenovirus as described in Curiel, Hum. Gene Ther. 3:147-154 (1992), may be employed.

Other gene delivery vehicles and methods may be employed, including polycationic condensed DNA linked or unlinked to killed adenovirus alone, for example, Curiel, Hum. Gene Ther. 3:147-154 (1992); ligand-linked DNA, for example, see Wu, J. Biol. Chem. 264:16985-16987 (1989); eukaryotic cell delivery vehicles cells, for example see U.S. Ser. No. 08/240,030, filed May 9, 1994, and U.S. Ser. No. 08/404,796; deposition of photopolymerized hydrogel materials; hand-held-gene transfer particle gun, as described in U.S. Pat. No. 5,149,655; ionizing radiation as described in U.S. Pat. No. 5,206,152 and in WO 92/11033; nucleic charge neutralization or fusion with cell membranes. Additional approaches are described in Philip, Mol. Cell Biol. 14:2411-2418 (1994) and in Woffendin, Proc. Natl. Acad. Sci. 91:1581-1585 (1994).

Naked DNA may also be employed. Exemplary naked DNA introduction methods are described in WO 90/11092 and U.S. Pat. No. 5,580,859. Uptake efficiency may be improved using biodegradable latex beads. DNA coated latex beads are efficiently transported into cells after endocytosis initiation by beads. The method may be improved further by treatment of the beads to increase hydrophobicity and thereby facilitate disruption of the endosome and release of the DNA into the cytoplasm. Liposomes that can act as gene delivery vehicles are described in U.S. Pat. No. 5,422,120, PCT Patent Publication Nos. WO 95/13796, WO 94/23697 and WO 91/14445, and EP No. 0 524 968.

Further non-viral delivery systems suitable for use include mechanical delivery systems such as the approach described in Woffendin et al., Proc. Natl. Acad. Sci. USA 91(24):11581-11585 (1994). Moreover, the coding sequence and the product of expression of such can be delivered through deposition of photopolymerized hydrogel materials. Other conventional methods for gene delivery that can be used for delivery of the coding sequence include, for example, use of hand-held gene transfer particle gun, as described in U.S. Pat. No. 5,149,655; use of ionizing radiation for activating transferred gene, as described in U.S. Pat. No. 5,206,152 and PCT Patent Publication No. WO 92/11033.

The present invention also relates to a method of stimulating cell proliferation, comprising administering an effective amount of FGF-9 (e.g., Kanda et al., supra.) FGF-20 or FGF-23, or a biologically-active fragment thereof. By the phrase “stimulating cell proliferation,” it is meant that the administered FGF results in cell division or mitosis. The FGF can be administered in any effective form (nucleic acid or polypeptide) to any suitable host.

For instance, in one embodiment, the method is useful to identify agonists and antagonists of FGF. In such cases, it can be useful to administer the FGF to cell lines, including established and primary cells, such as spinal motoneurons. Established lines include, e.g., any of the cell-lines stored at the American Tissue Culture Collection (atccc.org) including, e.g., DBTRG-05MG, PFSK-1, MSTO-211H, NCI-H378, NCI-N417, NCI-H526, HCN-1A, HCN-2, CATH.a, NG108-15, NCI-H446, NCI-H209, NCI-H146, NCI-H82, NCI-H345, NCI-H510A, D283 Med, D341 Med, C6, IMR-32, Neuro-2a, NB41A3, BC3H1, A172, Mpf, T98G[T98-G], SCP, CCF-STTG1, DI TNC1, CTX TNA2, PG4 (S+L-), G355-5, SW 598 [SW-598; SW598], C6/LacZ, 9L/lacZ, N1E-115, SH-SY5Y, BE(2)-M17, BE(2)-C, MC-IXC, SK-N-BE(2), CHP-212, C6/lacZ7, M059K, M059J, F98, RG2[D74], NCI-H250, NCI-H1915, OA1, TE 615.T, SVG p12, TE671 subline No. 2, MBr Cl 1, SK-N-MC, SW 1088 [SW-1088; SW1088], SW 1783 [SW-1783; SW1783], U-87 MG, U-118 MG, U-138 MG, MDA-MB-361, DU 145, Hs 683, H4, 293, PC-12, P19, NTERA-2 cl.D1[NT2/D1], BCE C/D-1b, SK-N-AS, SK-N-FI, SK-N-DZ, SK-N-SH, Daoy, preferably, N20.1 cells.

Putative agonists and antagonists of FGF can be administered in vitro to cells to which FGF has been administered, such as the cell lines described above, or the putative agents can be administered in vitro or in vivo to cells which naturally produce FGF. The agonistic or antagonistic effect of such agents can be measured with any of a variety of art-recognized assays, such as those described elsewhere herein.

Neural stem cells can also be stimulated to proliferate by an FGF of the present invention. The resulting cells can be used as a source of neural cells for transplantation back into same patient from which they were derived (i.e., autologous), eliminating any the classic problems associated with allogenic transplantation, such as rejection. Thus, a method of the present invention relates to administering an amount of FGF effective to stimulate proliferation and differentiation of neural stem cells, and transplanting said stem cells back

The present invention also relates to antibodies which specifically recognize an FGF of the present invention. An antibody specific for FGF means that the antibody recognizes a defined sequence of amino acids within or including an FGF, e.g., the sequence of FIGS. 1 and 2. Thus, a specific antibody will generally bind with higher affinity to an amino acid sequence, i.e., an epitope, found in FIGS. 1 and 2 than to a different epitope(s), e.g., as detected and/or measured by an immunoblot assay or other conventional immunoassay. Thus, an antibody which is specific for an epitope of human FGF-21 is useful to detect the presence of the epitope in a sample, e.g., a sample of tissue containing human FGF-21 gene product, distinguishing it from samples in which the epitope is absent. Such antibodies are useful as described in Santa Cruz Biotechnology, Inc., Research Product Catalog, and can be formulated accordingly.

Antibodies, e.g., polyclonal, monoclonal, recombinant, chimeric, humanized, can be prepared according to any desired method. See, also, screening recombinant immunoglobulin libraries (e.g., Orlandi et al., Proc. Natl. Acad. Sci., 86:3833-3837, 1989; Huse et al., Science, 256:1275-1281, 1989); in vitro stimulation of lymphocyte populations; Winter and Milstein, Nature, 349: 293-299, 1991. For example, for the production of monoclonal antibodies, a polypeptide according to FIGS. 1 and 2 can be administered to mice, goats, or rabbits subcutaneously and/or intraperitoneally, with or without adjuvant, in an amount effective to elicit an immune response. The antibodies can also be single chain or FAb fragments. The antibodies can be IgM, IgG, subtypes, IgG2a, IgG1, etc. Antibodies, and immune responses, can also be generated by administering naked DNA See, e.g., U.S. Pat. Nos. 5,703,055; 5,589,466; 5,580,859.

FGF, or fragments thereof, for use in the induction of antibodies do not need to have biological activity; however, they must have immunogenic activity, either alone or in combination with a carrier. Peptides for use in the induction of FGF-specific antibodies may have an amino sequence consisting of at least five amino acids, preferably at least 10 amino acids. Short stretches of FGF amino acids, e.g., five amino acids, can be fused with those of another protein such as keyhole limpet hemocyanin, or another useful carrier, and the chimeric molecule used for antibody production. Regions of FGF useful in making antibodies can be selected empirically, or, e.g., an amino acid sequence of GENE, as deduced from the cDNA, can be analyzed to determine regions of high immunogenicity. Analysis to select appropriate epitopes is described, e.g., by Ausubel FM et al (1989, Current Protocols in Molecular Biology, Vol 2. John Wiley & Sons).

Useful sequences for generating antibodies, include, the aligned sequences shown in FIGS. 1 and 2. Antibodies to such sequences can be useful for distinguishing between the different transcripts of FGF. See, above.

Particular FGF antibodies are useful for the diagnosis of prepathologic conditions, and chronic or acute diseases which are characterized by differences in the amount or distribution of FGF. Diagnostic tests for FGF include methods utilizing the antibody and a label to detect FGF in human (or mouse, etc, if using mouse, etc.) body fluids, tissues or extracts of such tissues.

The polypeptides and antibodies of the present invention may be used with or without modification. Frequently, the polypeptides and antibodies will be labeled by joining them, either covalently or noncovalently, with a substance which provides for a detectable signal. A wide variety of labels and conjugation techniques are known and have been reported extensively in both the scientific and patent literature. Suitable labels include radionuclides, enzymes, substrates, cofactors, inhibitors, fluorescent agents, chemiluminescent agents, magnetic particles and the like. Patents teaching the use of such labels include U.S. Pat. Nos. 3,817,837; 3,850,752; 3,939,350; 3,996,345; 4,277,437; 4,275,149; and 4,366,241.

Antibodies and other ligands which bind FGF can be used in various ways, including as therapeutic, diagnostic, and commercial research tools, e.g., to quantitate the levels of FGF polypeptide in animals, tissues, cells, etc., to identify the cellular localization and/or distribution of it, to purify it, or a polypeptide comprising a part of it, to modulate the function of it, in Western blots, ELISA, immunoprecipitation, RIA, etc. The present invention relates to such assays, compositions and kits for performing them, etc. Utilizing these and other methods, an antibody according to the present invention can be used to detect FGF polypeptide or fragments thereof in various samples, including tissue, cells, body fluid, blood, urine, cerebrospinal fluid.

In addition, ligands which bind to an FGF polypeptide according to the present invention, or a derivative thereof, can also be prepared, e.g., using synthetic peptide libraries or aptamers (e.g., Pitrung et al., U.S. Pat. No. 5,143,854; Geysen et al., J. Immunol. Methods, 102:259-274, 1987; Scott et al., Science, 249:386, 1990; Blackwell et al., Science, 250:1104, 1990; Tuerk et al., 1990, Science, 249: 505.).

The present invention also relates to an FGF polypeptide, prepared according to a desired method, e.g., as disclosed in U.S. Pat. No. 5,434,050. A labeled polypeptide can be used, e.g., in binding assays, such as to identify substances that bind or attach to FGF, to track the movement of FGF in a cell, in an in vitro, in vivo, or in situ system, etc.

A nucleic acid, polypeptide, antibody, ligand etc., according to the present invention can be isolated. The term “isolated” means that the material is in a form in which it is not found in its original environment or in nature, e.g., more concentrated, more purified, separated from component, etc. An isolated nucleic acid includes, e.g., a nucleic acid having the sequence of FGF separated from the chromosomal DNA found in a living animal, e.g., as the complete gene, a transcript, or a cDNA. This nucleic acid can be part of a vector or inserted into a chromosome (by specific gene-targeting or by random integration at a position other than its normal position) and still be isolated in that it is not in a form which it is found in its natural environment. A nucleic acid or polypeptide of the present invention can also be substantially purified. By substantially purified, it is meant that nucleic acid or polypeptide is separated and is essentially free from other nucleic acids or polypeptides, i.e., the nucleic acid or polypeptide is the primary and active constituent.

The present invention also relates to a transgenic animal, e.g., a non-human-mammal, such as a mouse, comprising an FGF. Transgenic animals can be prepared according to known methods, including, e.g., by pronuclear injection of recombinant genes into pronuclei of 1-cell embryos, incorporating an artificial yeast chromosome into embryonic stem cells, gene targeting methods, embryonic stem cell methodology. See, e.g., U.S. Pat. Nos. 4,736,866; 4,873,191; 4,873,316; 5,082,779; 5,304,489; 5,174,986; 5,175,384; 5,175,385; 5,221,778; Gordon et al., Proc. Natl. Acad. Sci., 77:7380-7384, 1980; Palmiter et al., Cell, 41:343-345, 1985; Palmiter et al., Ann. Rev. Genet., 20:465-499, 1986; Askew et al., Mol. Cell. Bio., 13:4115-4124, 1993; Games et al. Nature, 373:523-527, 1995; Valancius and Smithies, Mol. Cell. Bio., 11: 1402-1408, 1991; Stacey et al., Mol. Cell. Bio., 14:1009-1016, 1994; Hasty et al., Nature, 350:243-246, 1995; Rubinstein et al., Nucl. Acid Res., 21:2613-2617,1993. A nucleic acid according to the present invention can be introduced into any non-human mammal, including a mouse (Hogan et al., Manipulating the Mouse Embryo: A Laboratory Manual, Cold Spring Harbor Laboratory, Cold Spring Harbor, N.Y., 1986), pig (Hammer et al., Nature, 315:343-345, 1985), sheep (Hammer et al., Nature, 315:343-345, 1985), cattle, rat, or primate. See also, e.g., Church, 1987, Trends in Biotech. 5:13-19; Clark et al., Trends in Biotech. 5:20-24, 1987); and DePanphilis et al., BioTechniques, 6:662-680, 1988). In addition, e.g., custom transgenic rat and mouse production is commercially available. These transgenic animals can useful animals models to test for GENE function, as food for a snake, as a genetic marker to detect strain origin (i.e., where an FGF-21, -23, or fragment thereof has, been inserted), etc. Such transgenic animals can further comprise other transgenes. Transgenic animals can be prepared and used according to any suitable method.

For other aspects of the nucleic acids, reference is made to standard textbooks of molecular biology. See, e.g., Davis et al., Basic Methods in Molecular Biology, Elsevir Sciences Publishing, Inc., New York, 1986; Hames et al., Nucleic Acid Hybridization, IL Press, 1985; Sambrook et al., Molecular Cloning, CSH Press, 1989; Howe, Gene Cloning and Manipulation, Cambridge University Press, 1995.

### BRIEF DESCRIPTION OF THE DRAWINGS

FIG. 1 shows the nucleotide and amino acid sequence of FGF-20. (SEQ ID NOS. 1 and 2)

FIG. 2 shows the nucleotide and amino acid sequence of FGF-23. (SEQ ID NOS. 3 and 4)

FIG. 3 shows the aligned amino acid sequence of FGF-20 protein with known FGF-family members. xfgf-20 is from Xenopus laevis.

FIG. 4 shows oligodendrocyte proliferation. FIG. 4A shows the proliferation of oligodendrocytes. FIG. 4B shows that activity is abolished by boiling the protein.

FIG. 5 shows the effect of FGF-20 on N20.1 oligodendrocyte proliferation.

FIG. 6 shows the effect of FGFs on the proliferation of primary rat oligodendrocytes (PRO). FIG. 6A shows cells treated with FGF-2. FIG. 6B shows cells treated with FGF-20.

FIG. 7 shows the effect of FGFs on the survival/proliferation of a cell line of neuronal origin. FIG. 7A shows the effect of FGF-20. FIG. 7B shows the effect of FGF-2, FGF-9 and FGF-20.

FIG. 8 shows neurite outgrowth. Cultured PC12 cells are treated for 6 days with recombinant FGF-20 plus heparin (left panel) or heparin alone (right panel). Cells are fixed and stained for βIII-tubulin, nuclei are imaged with 7-AAD. Neurite outgrowth is not observed in cells treated with heparin alone.

FIG. 9 shows that FGF-20 is a potent survival factor for cortical neurons.

### EXAMPLES

### Example 1

Oligodendrocyte Proliferation and Survival:

Oligodendrocytes used for measuring the effects of growth factors (GF) on cell proliferation are either established cell lines such as N20 or primary rodent oligodendrocytes. Primary rodent (rat) oligodendrocytes and oligodendrocyte progenitors are isolated and purified by differential adhesion technique (Mitrovic, 1994) and Percol gradient centrifugation (Mattera, et al., Neurochem. Int. 1984, 6(1) 41-50; Kim, et al., J Neurol Sci December 1983:62(1-3): 295-301). Oligodendrocyte proliferation assays are carried out by plating 2.5×10⁴ cells/ml in 96 well plates. Cells are stimulated with growth factors for time periods of 3, 5 and 7 days. Positive controls are other members of FGF family such as FGF-2 or FGF-9. Cell proliferation is measured by MTT assay and ³H-Thymidine incorporation assay.

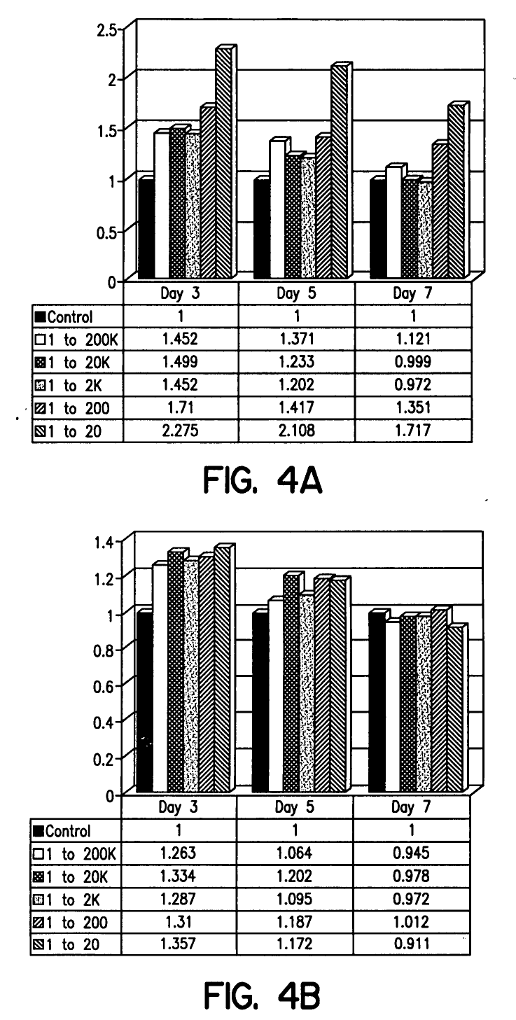

FIGS. 4, 5 and 6 show that FGF 20 stimulates oligodendrocyte proliferation of N 20.1 oligodendrocyte cell line in a time and dose responsive manner. N20.1 cells are treated with partially purified heparin agarose chromatography samples of FGF-20. Proliferation is determined by MTT staining. FGF-20 induces the proliferation of the oligodendrocytes (FIG. 4A) and the activity is abolished by boiling the protein (FIG. 4B).

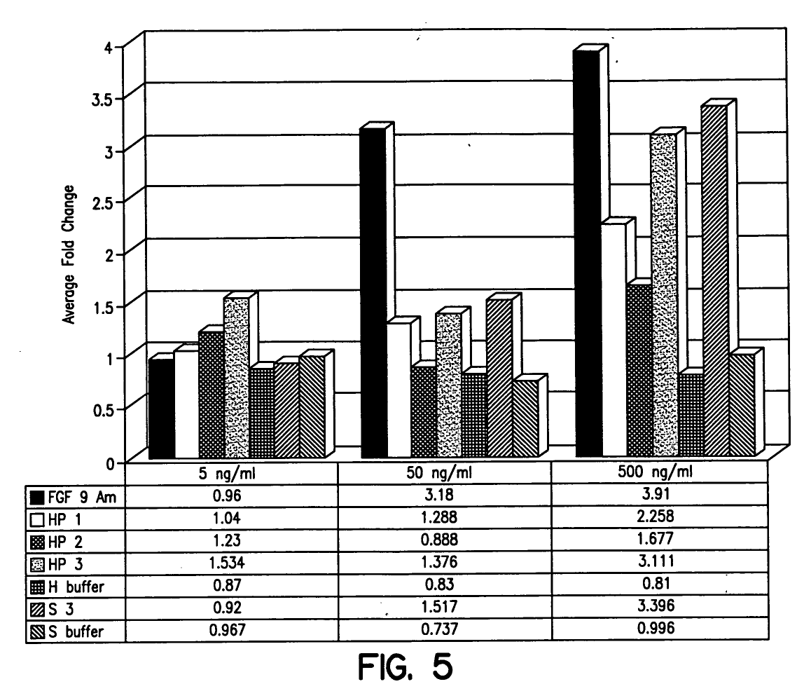

The above observations are confirmed with preparations of partially purified material from the heparin and S columns (FIG. 5). N20.1 cells are treated with FGF-20 from heparin or S columns. The cells are incubated with the FGF-20 for 5 days and the increase in proliferation over non-treated control is determined by MTT staining. FGF-9 is used as a positive control, and appropriate corresponding buffers (H and S) are used as a negative control. The activity of a partially purified material is comparable to FGF-9.

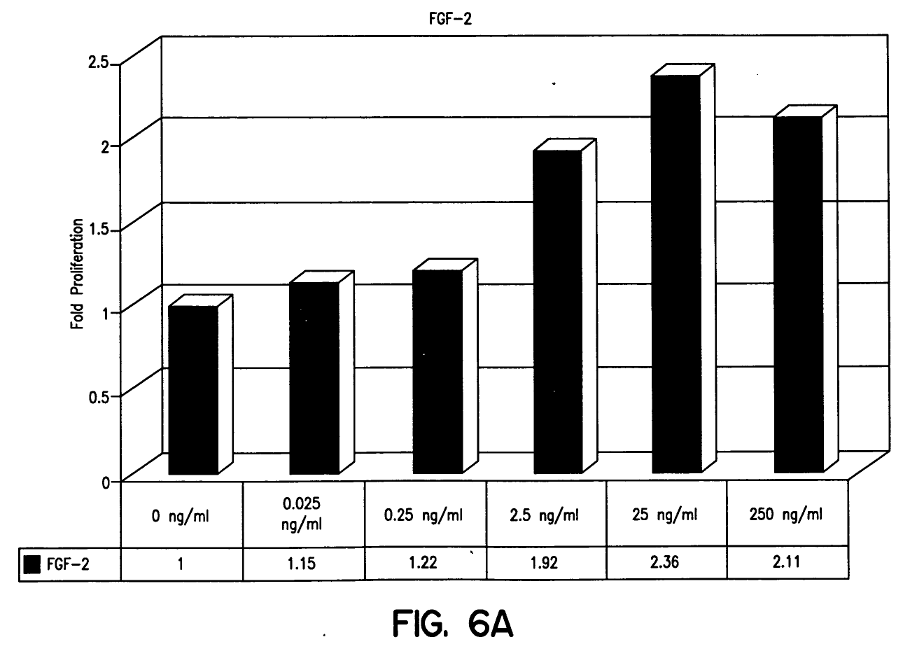

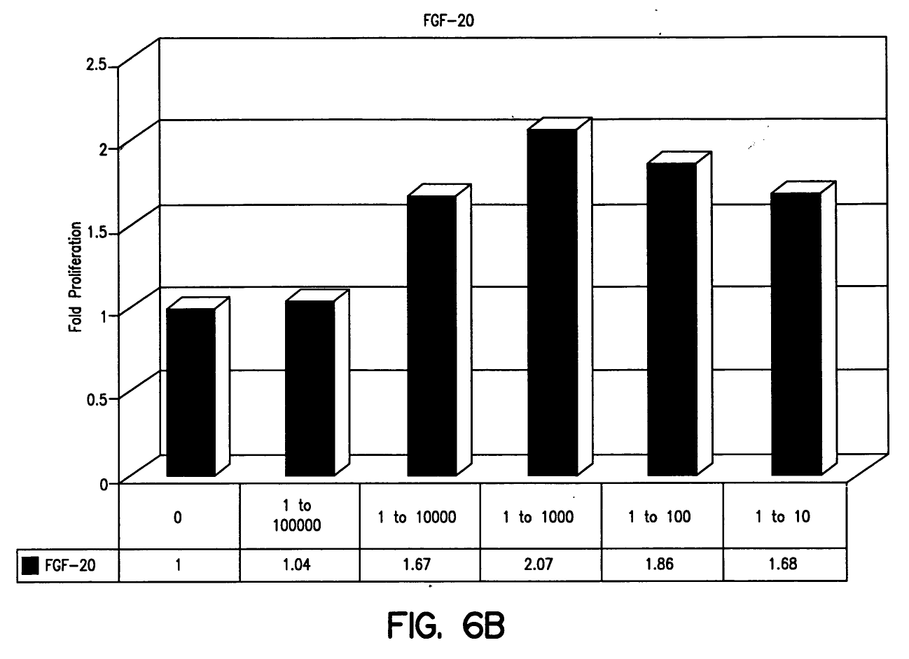

Furthermore, FGF-20 induces the proliferation of primary rat oligodendrocytes (FIG. 6B). Oligodendrocytes are treated with FGF-2 (FIG. 6A), and FGF-20 (FIG. 6B). The cells are incubated with the GFs for 3 days and the increase in proliferation over non-treated control is determined by MTT staining. The activity of a partially purified material is comparable to FGF-2. FGF-20 is a potent inducer of oligodendrocyte proliferation and its activity is comparable with other members of FGF family such as FGF-2 and FGF-9.

### Example 2

Induction of Neuronal Survival:

Neuronal survival assays are carried out by plating 2.5×10⁴ cells/ml in 96 well plates in low serum media. Under these conditions neuronal cells undergo apoptosis due to the growth factor withdrawal. Cells are stimulated with growth factors for different time periods ranging from 3 days to 12 days. Positive controls are other members of FGF family such as FGF-2 or FGF-9. Neuronal survival is measured by MTT.

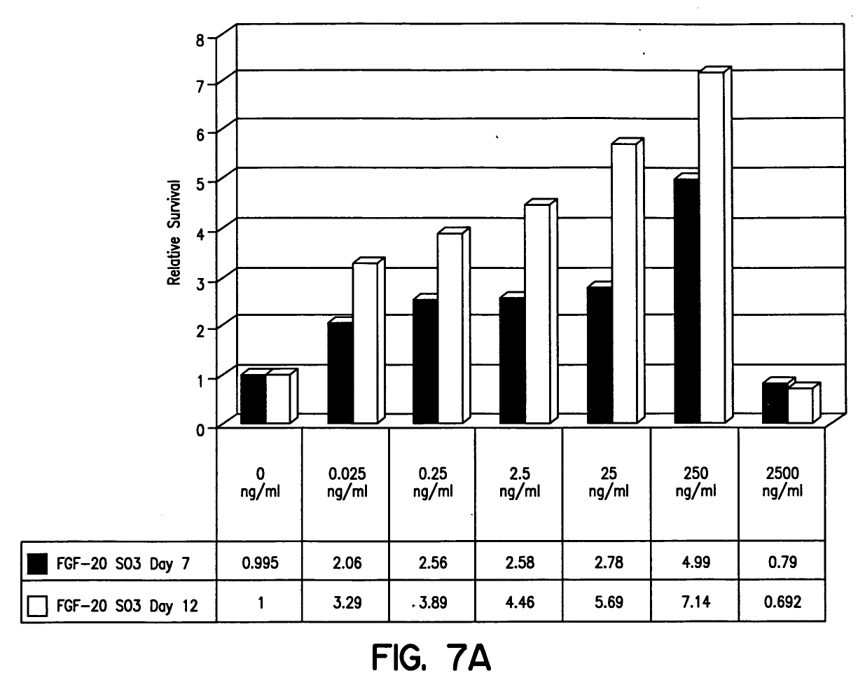

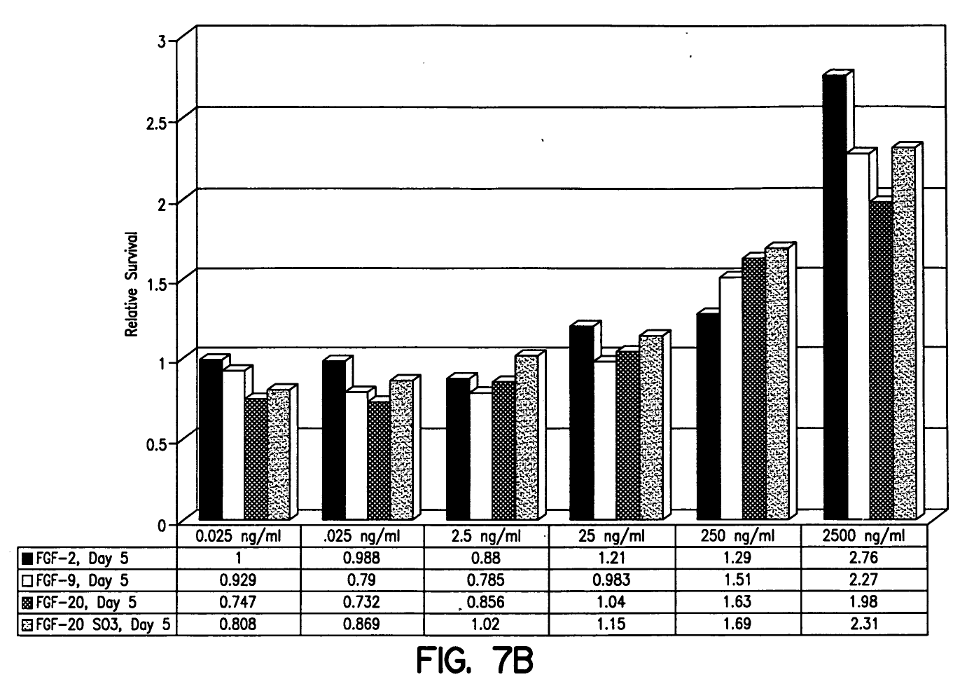

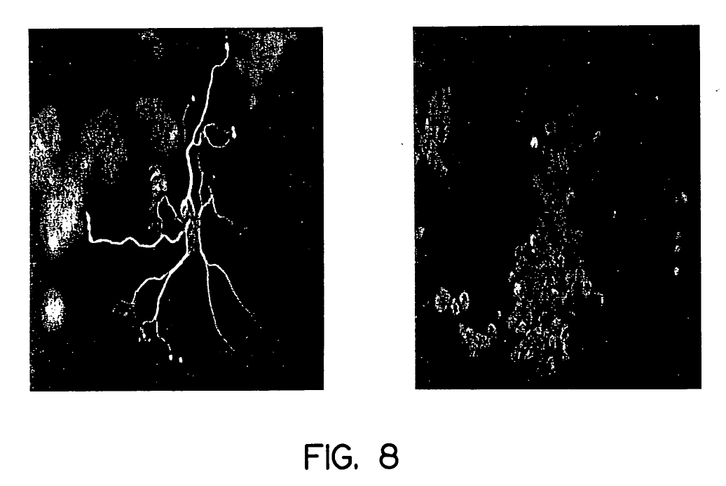

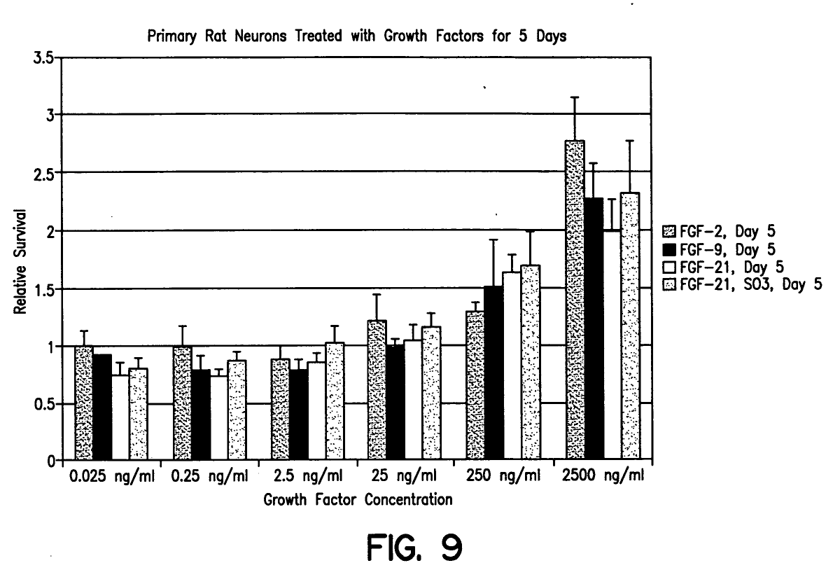

FIGS. 7 and 9 show that FGF-20 is a potent neurotrophic factor which can stimulate the survival of the cells of neuronal origin.

PC 12 cells are plated in 96 well plates in the presence of low serum media (1% Nu serum). Different growth factors, including FGF-20 are added in concentrations ranging from 0.0025-2500 ngs/ml. 7 and 10 days after, relative survival is measured with MTT assay and compared with non treated control. Data for FGF-20 are shown in FIG. 7A, and for FGF-2, FGF-9 and FGF-20 in FIG. 7B.

### Example 3

Induction of Neurite Outgrowth

FGF-20 exhibits activity on the outgrowth of PC 12 cells. This activity is not dependent on NGF pretreatment (see Tables 1 and 2 and FIG. 9).

The behavior of the partially purified FGF-21 in this assay is similar to that observed for FGF-9 to which it is very similar in sequence. In addition, the activity of different members of FGF family on in the induction of neurite outgrowth in PC 12 cells (FGF-1, FGF-2, FGF-4, FGF-6, FGF-7, FGF-8, FGF-9, FGF-10, FGF-16, FGF-16, FGF-17, FGF-18—(see Table 2) are compared. The most potent FGFS in inducing neurite outgrowth in this system are FGF-2 and FGF-9 and FGF-20/21. Two FGFs, FGF-7 and FGF-10, are found to be inactive in this assay regardless of the presence or absence of heparin.

Primary rat fetal cortical neurons are isolated from embrionic rat brains (E16). The cortex is dissected under the microscope and washed 6 times with Hanks solution and mechanically dissociated without the enzymatic treatment. Neurons are cultured in a medium consisting of the following: DMEM supplemented with 10% horse serum, 10% FCS, 2 mM L-glutamine, HEPES buffer. After 24 h a cocktail of inhibitors consisting of 10 uM FdU and 1 uM cytosine arabinoside is added for 3 days in order to inhibit the proliferation of all other cell types except neurons. After 8 days in culture, neurons are harvested and plated in 96 well plates in the presence of low serum media (2% Nu serum). Different growth factors, including FGF-20 are added in concentrations ranging from 0.0025-2500 ngs/ml. After 5 days, relative survival is measured with MTT assay and compared to non-treated control.

PC 12 cells are plated and treated as in the experiment shown in FIG. 7. FGF-9 and FGF-20 are added in the concentrations ranging from 0.0025-2500 ngs/ml. Seven and 12 days after the treatment the neurite extension is determined by staining the cells with Wright stain and subsequent microscopic examination. The % outgrowth represents the estimated number of the cells with processes.

The summary of the observations for the induction of neurite outgrowth due to FGF-9 and FGF-20/21 treatments are shown below. The highest concentration of partially purified material is toxic to the cells, which affects both the survival data (See FIG. 7B) and the neurite outgrowth (see below).

TABLE 1
FGF-20 is a potent inducer of the neurite extensions in PC12 cells:
Neurite Extension in PC 12 cells

| | |
|---|---|
| concentration | % outgrowth |

| GF | ngs/ml | 7 days | 12 days |
|---|---|---|---|
| FGF-9 | 0 | 0 | 0 |
| | 0.025 | <5 | 5 |
| | 0.25 | 5 | 10-20 |
| | 2.5 | 5-10 | 20-30 |
| | 25 | 60 | 60 |
| | 250 | 90 | 100 |
| | 2500 | 90-100 | 100 |
| FGF-21 | 0 | 0 | 0 |
| | 0.025 | <5 | 5 |
| | 0.25 | 10 | 10 |
| | 2.5 | 20-30 | 30 |
| | 25 | 50 | 60 |
| | 250 | 90 | 80 |
| | 2500 | 0 | 0 |

TABLE 3
Neurite outgrowth-Comparison of different FGF family members:
Cultured PC12 cells are treated with FGFs and neurite outgrowth
is scored visually. FGF-20 is one of the most potent neurotrophic
GF from FGF family members tested..

| FGF Added: | Response |
|---|---|
| FGF-1 (acidic FGF) | ++ |
| FGF-2 (basic FGF) | ++++ |
| FGF-4 | + |
| FGF-6 | + |
| FGF-7 | − |
| FGF-8 | ++ |
| FGF-9 | +++ |
| FGF-10 | − |
| FGF-16 | + |
| FGF-17 | ++ |
| FGF-18 | ++ |
| FGF-21 | +++ |

We claim:

1. A method to treat spinal cord damage; spinal cord trauma; neuronal tissue damage produced by an ischemic attack, infarction, hemorrhage or aneurysm; Huntington's disease; myelopathy; myelitis; or syringomyelia, comprising administering to a patient in need thereof an effective amount of an FGF-20 polypeptide or a biologically active fragment thereof.

2. The method of claim 1, wherein said FGF-20 polypeptide is human.

3. The method of claim 2, wherein said polypeptide has FGF-20 specific immunogenic activity.

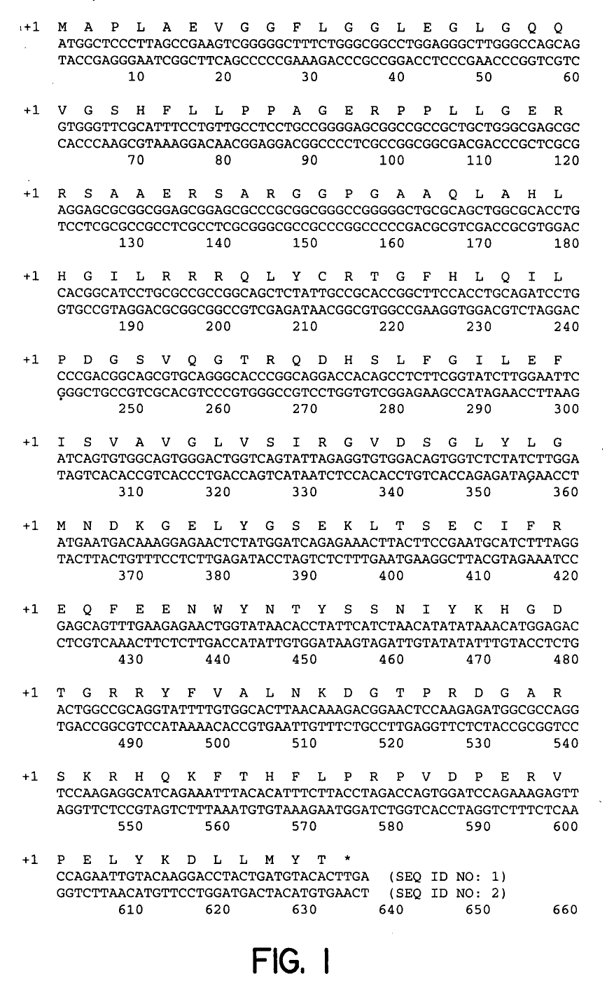

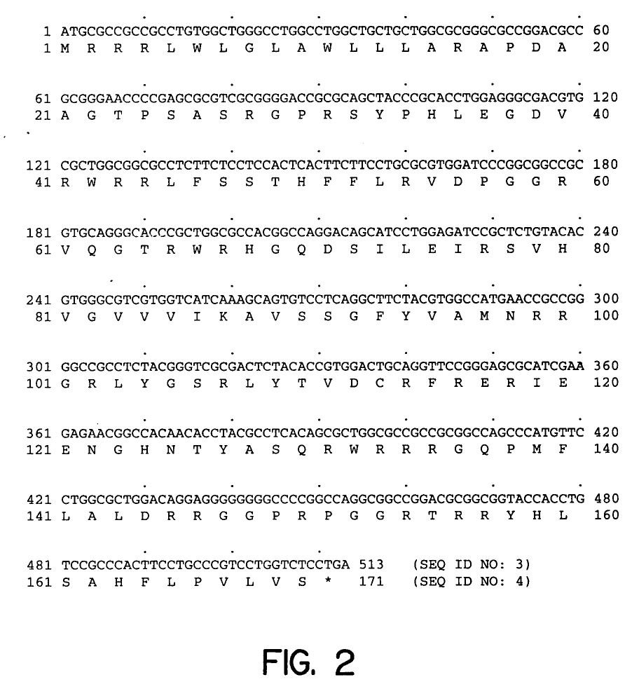

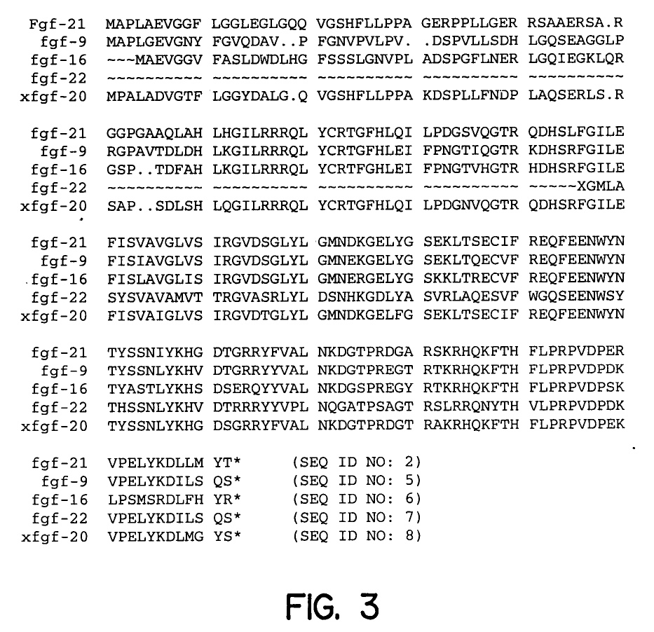

4. The method of claim 1, wherein said polypeptide comprises amino acid 1 to amino acid 211 as set forth in FIG. 1.

5. The method of claim 1, wherein said polypeptide has 95% sequence identity to amino acid 1 to amino acid 211 of human FGF-20 as set forth in FIG. 1, and wherein said FGF-20 has FGF activity.

6. The method of claim 2,wherein said polypeptide has 95% sequence identity to amino acid 1 to amino acid 211 of human FGF-20 as set forth in FIG. 1, and wherein said FGF-20 has FGF activity.

7. A method to treat spinal cord damage; spinal cord trauma; neuronal tissue damage produced by an ischemic attack, infarction, hemorrhage or aneurysm; Huntington's disease; myelopathy; myelitis; or syringomyelia, comprising administering to a patient in need thereof an effective amount of a nucleic acid having a nucleotide sequence coding for an FGF-20 polypeptide or a biologically active fragment thereof.

8. The method of claim 7, wherein said nucleic acid is human.

9. The method of claim 8, wherein the nucleotide sequence codes without interruption for FGF-20.

10. The method of claim 7, wherein the nucleotide sequence has 95% sequence identity to the nucleotide sequence set forth in FIG. 1.

11. The method of claim 8, wherein the nucleotide sequence has 95% sequence identity to the nucleotide sequence set forth in FIG. 1.

12. A method to treat an adrenal leukodystrophy, progressive multifocal leukoencephalopathy, encephalomyelitis, Guillian-Barre syndrome, paraproteinemia, or chronic inflammatory demyelinating polyneuropathy, comprising administering to a patient in need thereof an effective amount of a nucleic acid having a nucleotide sequence coding for an FGF-20 polypeptide or a biologically active fragment thereof.

13. The method of claim 12, wherein said FGF-20 polypeptide is human.

14. The method of claim 13, wherein said polypeptide has FGF-20 specific immunogenic activity.

15. The method of claim 12, wherein said polypeptide comprises amino acid 1 to amino acid 211 as set forth in FIG. 1.

16. The method of claim 12, wherein said polypeptide has 95% sequence identity to amino acid 1 to amino acid 211 of human FGF-20 as set forth in FIG. 1, and wherein said FGF-20 has FGF activity.

17. The method of claim 13, wherein said polypeptide has 95% sequence identity to amino acid 1 to amino acid 211 of human FGF-20 as set forth in FIG. 1, and wherein said FGF-20 has FGF activity.

18. A method to treat an adrenal leukodystrophy, progressive multifocal leukoencephalopathy, encephalomyelitis, Guillian-Barre syndrome, paraproteinemia, or chronic inflammatory demyelinating polyneuropathy, comprising administering to a patient in need thereof an effective amount of a nucleic acid having a nucleotide sequence coding for an FGF-20 polypeptide or a biologically active fragment thereof.

19. The method of claim 18, wherein said nucleic acid is human.

20. The method of claim 19, wherein the nucleotide sequence codes without interruption for FGF-20.

21. The method of claim 18, wherein the nucleotide sequence has 95% sequence identity to the nucleotide sequence set forth in FIG. 1.

22. The method of claim 19, wherein the nucleotide sequence has 95% sequence identity to the nucleotide sequence set forth in FIG. 1.

23. A method to promote graft survival, comprising administering to a patient in need thereof an effective amount of an FGF-20 polypeptide or a biologically active fragment thereof.

24. The method of claim 23, wherein said FGF-20 polypeptide is human.

25. The method of claim 24, wherein said polypeptide has FGF-20 specific immunogenic activity.

26. The method of claim 23, wherein said polypeptide comprises amino acid 1 to amino acid 211 as set forth in FIG. 1.

27. The method of claim 23, wherein said polypeptide has 95% sequence identity to amino acid 1 to amino acid 211 of human FGF-20 as set forth in FIG. 1, and wherein said FGF-20 has FGF activity.

28. The method of claim 24, wherein said polypeptide has 95% sequence identity to amino acid 1 to amino acid 211 of human FGF-20 as set forth in FIG. 1, and Wherein said FGF-20 has FGF activity.

29. A method to promote graft survival, comprising administering to a patient in need thereof an effective amount of a nucleic acid having a nucleotide sequence coding for an FGF-20 polypeptide or a biologically active fragment thereof.

30. The method of claim 29, wherein said nucleic acid is human.

31. The method of claim 30, wherein the nucleotide sequence codes without interruption for FGF-20.

32. The method of claim 29, wherein the nucleotide sequence has 95% sequence identity to the nucleotide sequence set forth in FIG. 1.

33. The method of claim 30, wherein the nucleotide sequence has 95% sequence identity to the nucleotide sequence set forth in FIG. 1.
